(19) United States
(12) Patent Application Publication
(10) Pub. No.: US 2013/0244995 A1
(43) Pub. Date: Sep. 19, 2013

(54) CALCIUM-SENSING RECEPTOR-ACTIVE COMPOUNDS

(71) Applicants: Per Vedsø, Ballerup (DK); Lars Kristian Albert Blæhr, Ballerup (DK)

(72) Inventors: Per Vedsø, Ballerup (DK); Lars Kristian Albert Blæhr, Ballerup (DK)

(73) Assignee: LEO PHARMA A/S, Ballerup (DK)

(21) Appl. No.: 13/989,443

(22) PCT Filed: Nov. 21, 2011
(86) PCT No.: PCT/EP2011/070574

§ 371 Date: Jun. 4, 2013

Related U.S. Application Data
(60) Provisional application No. 61/417,373, filed on Nov. 26, 2010.

(51) Int. Cl. C07D 295/192; C07D 211/58; C07C 237/20; C07D 205/04; C07D 241/08; C07C 317/28

(52) U.S. Cl. CPC C07D 295/192; C07D 241/08; C07C 317/28; C07C 237/20; C07D 205/04; C07D 211/58
USPC 514/210.17; 544/384; 514/255.01; 564/99; 560/51; 514/605; 564/86; 514/603; 564/165; 514/620; 548/952; 540/575; 514/218; 546/223; 514/329; 544/391; 560/45; 562/452; 549/464; 562/456

(57) ABSTRACT

Compounds of general formula (I), their use as calcium receptor-active compounds for the prophylaxis, treatment or amelioration of physiological disorders or diseases associated with disturbances of CaSR activity, such as hyperparathyroidism, pharmaceutical compositions comprising said compounds, and methods of treating diseases with said compounds.

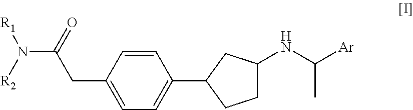

### FIELD OF THE INVENTION

This invention relates to novel calcium-sensing receptor-active compounds, to said compounds for use in therapy, to pharmaceutical compositions comprising said compounds, to methods of treating diseases with said compounds, and to the use of said compounds in the manufacture of medicaments.

### BACKGROUND OF THE INVENTION

The calcium-sensing receptor (CaSR) is a G-protein-coupled receptor (GPCR) that signals through the activation of phospholipase C, increasing levels of inositol 1,4,5-triphosphate and cytosolic calcium. The CaSR belongs to the subfamily C of the GPCR superfamily, which also includes receptors for glutamate, gamma amino-butyric acid (GABA), pheromones and odorants that all possess a very large extra-cellular domain. This domain is highly negatively charged and is involved in binding of calcium and other positively charged molecules. The CaSR is found in the parathyroid glands but has also been identified in the brain, intestine, pituitary, thyroid glands, bone tissue and kidneys [Brown, E. M. Calcium-Sensing Receptor. Primer of the Metabolic Bone Diseases and Disorders of Mineral Metabolism Fifth Edition, 2003 by American Society for Bone and Mineral Research, Chapter 17, p. 111.; Drueke, T. E. Nephrol Dial Transplant (2004) 19, v20-v26].

The calcium sensing receptor (CaSR) detects changes in extra-cellular calcium concentration and initiates the functional response of this cell, which is a modulation of the secretion of the parathyroid hormone (PTH). Secretion of PTH increases extra-cellular calcium ion concentration by acting on various cells, such as bone and kidney cells, and the extra-cellular calcium ion concentration reciprocally inhibits the secretion of PTH by acting on parathyroid cells. The reciprocal relationship between calcium concentration and PTH level is an essential mechanism for calcium homeostasis maintenance.

The calcimimetic activity corresponds to the ability to produce or induce biological responses observed through variations in the concentration of extracellular calcium ions (Ca²⁺)ₑ and extracellular magnesium ions (Mg²⁺)ₑ.

(Ca²⁺)ₑ and (Mg²⁺)ₑ ions play a major role in the body through their regulation of calcium homeostasis on which many vital functions of the body depend. Thus, hypo- and hypercalcemia, that is to say conditions in which (Ca²⁺)ₑ ions are below or above the mean threshold, have a major effect on many functions, such as cardiac, renal or intestinal functions. They deeply affect the central nervous system (Chattopadhyay et al. Endocr. Review, Vol.17, 4, pp 289-307 (1996)).

It has been shown that Ca²⁺ and Mg²⁺ ions, but also Ba²⁺ ions, within millimolar concentration ranges, stimulate CaSRs. Activation of CaSRs might be induced in the brain by β-amyloid peptides, which are involved in neurodegenerative diseases such as Alzheimer's disease (Ye et al, J. Neurosci., 47, 547-554, Res. 1997).

Disturbance of CaSR activity is associated with biological disorders such as primary and secondary hyperparathyroidism, osteoporosis, cardiovascular, gastrointestinal, endocrine and neurodegenerative diseases, or certain cancers in which (Ca²⁺), ions are abnormally high.

Primary hyperparathyroidism (primary HPT) is characterised by elevated levels of PTH and serum calcium which is typically caused by adenoma of the parathyroid gland. It can result in bone pain and excessive bone resorption.

Secondary hyperparathyroidism (secondary HPT) often develops in patients who have reduced kidney function and is characterised by elevated levels of PTH. The underlying causes are complex, but a reduced ability to convert vitamin D to calcitriol and elevated levels of phosphorus play significant roles in the development of secondary HPT. If left untreated, the clinical manifestations of secondary HPT include bone and joint pain and limb deformities [Harrington, P. E. and Fotsch, C. Calcium Sensing Receptor Activators: Calcimimetics. Current Medicinal Chemistry, 2007, 14, 3027-3034].

A reduced kidney function or renal failure is also accompanied by renal osteodystrophy, e.g. osteitis fibrosa, osteomalacia, adynamic bone disease, or osteoporosis. These disorders are characterized by either high or low bone turnover. Osteoporosis is a multifactor disease which depends in particular on age and sex. While menopausal women are very greatly affected, osteoporosis is increasingly proving to be a problem in elderly men as well, and, for the moment, no optimal treatment exists. Its social cost may become even heavier in the years to come, particularly as life expectancy is becoming longer. Osteoporosis is currently treated with estrogens, calcitonin or biphosphonates which prevent bone resorption without stimulating bone growth. More recent data demonstrate that intermittent increases in PTH or in derivatives thereof are effective in the treatment of osteoporosis and make it possible to remodel bone by stimulating bone formation (Whitfield et al., Drugs & Aging, 15 (2) pp 117-129 (1999)). This new therapeutic approach for treatment of osteoporosis appears to be very advantageous, although major problems are associated with the use of PTH hormone, such as the route of injection, but also the appearance of tumors, observed recently during clinical trials in humans. Intermittent secretion of endogenous PTH can be obtained by blocking the calcium sensing receptor. The blocking of PTH secretion with CaSR agonists may be followed by a rapid increase in PTH (rebound effect), which is then beneficial in the treatment of osteoporosis.

A compound having an activating effect on CaSR (CaSR agonist), that is, a compound which selectively acts on CaSR to mimic or strengthen the action of Ca²⁺, is called a calcimimetic. On the other hand, a compound having an antagonistic effect on CaSR (CaSR antagonist, that is, a compound which suppresses or inhibits the action of Ca²⁺), is called a calcilytic.

The calcium-sensing receptor has recently been found to be a potent target for developing novel therapies such as using calcimimetics for treatment of diarrhea. [Osigweh et al, J American Coll. of Surgeons, V201, Issue 3, suppl 1, September 2005, p 17.]

Calcimimetics have been shown to be commercially useful for the treatment of hyperparathyroidism (HPT): The calcimimetic compound Cinacalcet® [Balfour, J. A. B. et al. Drugs (2005) 65(2), 271-281; Linberg et. al. J. Am. Soc. Nephrol (2005), 16, 800-807, Clinical Therapeutics (2005), 27(11), 1725-1751] is commercially available for the treatment of secondary HPT in chronic kidney disease patients on dialysis and for the treatment of primary HPT in patients with parathyroid carcinoma. Thus, proof of concept for activators of calcium sensing receptor (CaSR) in humans has been achieved and the clinical relevance is well established.

Other calcimimetic compounds are for example described in WO02/059102, WO98/001417, WO05/065050, WO 05/34928, WO03/099814, WO03/099776, WO00/21910, WO01/34562, WO01/090069, WO97/41090, U.S. Pat. No. 6,001,884, WO96/12697, EP1203761, WO95/11221, WO93/04373, EP1281702, WO02/12181, WO04/56365, WO04/069793, WO04/094362, US2004242602, WO04/106280, WO04/106295, WO04/106296, WO05/068433, WO05/115975, EP 1757582, WO 2009/051718, WO 2008/019690, WO 2009/065406 and WO2010/021351.

### SUMMARY OF THE INVENTION

The novel compounds of the present invention are modulators, e.g. activators or agonists of the human calcium sensing receptor (CaSR) and may thus be useful in the treatment or prophylaxis of a number of diseases or physiological disorders involving modulation of CaSR activity.

Accordingly, the present invention relates to a compound of general formula I

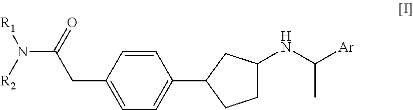

wherein

Ar represents phenyl or C₁₋₅heterocycloalkylphenyl, wherein said phenyl is optionally substituted with one or more, same or different substituents independently selected from halogen, hydroxy, C₁₋₆alkyl, trifluoromethyl or C₁₋₄alkoxy;

R₁ represents hydrogen, or is selected from the group consisting of C₁₋₆alkyl, C₂₋₆alkenyl, C₂₋₆alkynyl, hydroxyC₂₋₆alkyl, aminoC₂₋₆alkyl, hydroxyC₂₋₆alkylaminoC₂₋₆alkyl, C₁₋₃alkylsulfonylaminoC₂₋₆alkyl, aminosulfonylC₁₋₆alkyl, aminocarbonylC₁₋₆alkyl, or C₁₋₅heterocycloalkyl comprising 1-4 hetero atoms selected from N, O and S, wherein said C₁₋₆alkyl, C₂₋₆alkenyl, C₂₋₆alkynyl, hydroxyC₂₋₆alkyl, aminoC₂₋₆alkyl, hydroxyC₂₋₆alkylaminoC₂₋₆alkyl, C₁₋₃alkylsulfonylaminoC₂₋₆alkyl, aminosulfonylC₁₋₆alkyl, aminocarbonyIC₂₋₆alkyl, or C₁₋₅heterocycloalkyl comprising 1-4 hetero atoms selected from N, O and S, is optionally further substituted by one or more substituents selected from halogen, hydroxy, trifluoromethyl, C₁₋₃alkylsulfonylamino or —NH₂;

R₂ represents hydrogen, or is selected from the group consisting of C₁₋₆alkyl, C₂₋₆alkenyl, aminoC₂₋₆alkyl, C₃₋₇cycloalkyl, or C₁₋₅heterocycloalkyl comprising 1-4 hetero atoms selected from N, O and S;

provided one of R₁ and R₂ is not hydrogen;

or R₁ and R₂ together with the adjacent nitrogen to which they are attached form a 4, 5, 6 or 7-membered C₁₋₆heterocycloalkyl comprising one or more heteroatoms selected from the group consisting of O, S and N, said C₁₋₆heterocycloalkyl being optionally substituted by oxo, hydroxy, halogen, trifluoromethyl, C₁₋₆alkyl, —NH₂, —S(O)₂NH₂ , S(O)₂CH₃, C₁₋₆alkylcarbonyl, hydroxyC₂₋₆alkyl, C₁₋₆alkoxy, aminoC₂₋₆alkyl, C₁₋₆alkylamino, or aminosulfonylC₁₋₆alkylamino;

as well as stereoisomers, pharmaceutically acceptable salts, solvates, or hydrates thereof.

The compounds of the present invention may for example be useful in the treatment of complications associated with chronic kidney disease, such as hyperparathyroidism, e.g. primary and/or secondary hyperparathyroidism, or tertiary hyperparathyroidism. Other complications associated with chronic kidney disease are anemia, cardiovascular diseases, and the compounds of the present invention are also believed to have a beneficial effect on these diseases. The compounds of the present invention may furthermore be useful for promoting osteogenesis and treating or preventing osteoporosis, such as steroid induced, senile and post-menopausal osteoporosis; osteomalacia and related bone disorders, or for the prevention of bone loss post renal transplantation, or in rescue therapy pre-parathyroidectomy.

It is presently believed that the compounds of the present invention may have advantageous pharmacokinetic or pharmacodynamic properties, such as prolonged in vivo half-life and prolonged in vivo efficacy, in comparison to known structurally related compounds.

The compounds of formula I, Ia and Ib according to the present invention all contain features that imparts on the molecules a high stability towards human liver microsomes and hepatocytes, as well as increased volumes of distribution in vivo, which may render the compounds of the present invention especially suitable for intravenous or other parenteral administration.

In another aspect, the invention relates to the compound of general formula I, Ia or Ib as defined above for use as a medicament in therapy.

In another aspect, the invention relates to the compound of general formula I, Ia or Ib as defined above for use in the treatment, amelioration or prophylaxis of physiological disorders or diseases associated with disturbances of CaSR activity, such as hyperparathyroidism.

In yet another aspect, the invention relates to a pharmaceutical composition comprising a compound of formula I, Ia or Ib or a pharmaceutically acceptable salt, solvate, hydrate or in vivo hydrolysable ester thereof together with a pharmaceutically acceptable excipient or vehicle.

In a further aspect, the invention relates to a method of preventing, treating or ameliorating parathyroid carcinoma, parathyroid adenoma, primary parathyroid hyperplasia, cardiac, renal or intestinal dysfunctions, diseases of the central nervous system, chronic renal failure, chronic kidney disease, polycystic kidney disorder, podocyte-related diseases, primary hyperparathyroidism, secondary hyperparathyroidism, tertiary hyperparathyroidism, anemia, cardiovascular diseases, renal osteodystrophy, osteitis fibrosa, adynamic bone disease, osteoporosis, steroid induced osteoporosis, senile osteoporosis, post-menopausal osteoporosis, osteomalacia and related bone disorders, bone loss post renal transplantation, cardiovascular diseases, gastrointestinal diseases, endocrine and neurodegenerative diseases, cancer, Alzheimer's disease, IBS, IBD, malassimilation, malnutrition, abnormal intestinal motility such as diarrhea, vascular calcification, abnormal calcium homeostasis, hypercalcemia, or renal bone diseases, the method comprising administering to a patient in need thereof an effective amount of a compound of general formula I, Ia or Ib, optionally in combination or as supplement with an active vitamin-D sterol or vitamin-D derivative, such as 1-α-hydroxycholecalciferol, ergocalciferol, cholecalciferol, 25-hydroxycholecalciferol, 1-α-25-dihydroxycholecalciferol, or in combination or as supplement with phosphate binders, estrogens, calcitonin or biphosphonates.

In a still further aspect, the invention relates to intermediate compounds useful for the synthesis of compounds according to formula I, Ia or Ib.

### DETAILED DESCRIPTION OF THE INVENTION

Definitions

The term “aryl” is intended to indicate a radical of aromatic carbocyclic ring(s) comprising 6-10 carbon atoms, in particular 5- or 6-membered rings, optionally fused carbocyclic rings with at least one aromatic ring, such as phenyl, naphthyl.

The term “cycloalkyl” is intended to indicate a saturated cycloalkane radical or ring, comprising 3-7 carbon atoms, such as 3-6 carbon atoms, such as 4-5 or 5-6 carbon atoms, e.g. cyclopropyl, cyclobutyl, cyclopentyl, cyclohexyl and cycloheptyl.

The term “heterocycloalkyl” is intended to indicate a cycloalkyl radical as defined above, in particular 4, 5, 6 or 7-membered ring(s), such as 5-6 membered ring(s), in particular comprising 1-6 or 1-5 carbon atoms and 1-4 heteroatoms selected from O, N or S, such as 4-5 carbon atoms and 1-3 heteroatoms selected from O, N, or S, e.g. morpholino, morpholinyl, piperidyl, and piperazinyl.

The term “heterocycloalkylphenyl”, is intended to indicate a radical of a fused phenyl and heterocycloalkyl ring, said heterocycloalkyl as defined above, e.g. benzodioxolyl.

The term “halogen” is intended to indicate a substituent from the 7^{th} main group of the periodic table, preferably fluoro, chloro and bromo.

The term “alkyl” is intended to indicate the radical obtained when one hydrogen atom is removed from a hydrocarbon. Said alkyl comprises 1-6, preferably 1-4 or 1-3, such as 2-3, carbon atoms. The term includes the subclasses normal alkyl (n-alkyl), secondary and tertiary alkyl, such as methyl, ethyl, n-propyl, isopropyl, n-butyl, isobutyl, sec-butyl, tert-butyl, pentyl, isopentyl, hexyl and isohexyl.

The term “alkenyl” is intended to indicate a hydrocarbon radical comprising 1-4 C—C double bonds, e.g. 1, 2 or 3 double bonds and 2-6 carbon atoms, in particular 2-4 carbon atoms, such as 2-3 carbon atoms, e.g. ethenyl, allyl, propenyl, butenyl, pentenyl, hexenyl etc.

The term “alkynyl” is intended to indicate a hydrocarbon radical comprising 1-4 C—C triple bonds, e.g. 1, 2 or 3 triple bonds and 2-6 carbon atoms, in particular 2-4 carbon atoms, such as 2-3 carbon atoms, e.g. ethynyl, propynyl, butynyl, or pentynyl.

The term “hydroxyalkyl” is intended to indicate a radical of the formula ——OH, wherein R represents alkyl as indicated above, e.g. hydroxyethyl or hydroxypropyl.

The term “hydroxyalkylaminoalkyl” is intended to indicate a radical of the formula —R—NH—R′—OH, wherein R and R′ is alkyl as defined above, e.g. hydroxyethylaminoethyl etc.

The term “alkoxy” is intended to indicate a radical of the formula —OR, wherein R is alkyl as indicated above, e.g. methoxy, ethoxy, n-propoxy, isopropoxy, butoxy, etc.

The term “aminoalkyl” is intended to indicate a radical of the formula —R—NH₂, wherein R represents alkyl as indicated above, e.g. aminoethyl or aminopropyl.

The term “aminocarbonylalkyl” is intended to indicate a radical of the formula —R—C(O)—NH2, wherein R represents alkyl as indicated above, e.g. aminocarbonylmethyl, aminocarbonylethyl or aminocarbonylpropyl.

The term “alkylamino” is intended to indicate a radical of the formula —NH—R, wherein R represents alkyl as defined above, e.g. methylamino, ethylamino, or propylamino.

The term “alkylcarbonyl” is intended to indicate a radical of the formula —C(O)—R, wherein R represents alkyl as defined above, e.g. methylcarbonyl, or ethylcarbonyl.

The term “alkylaminocarbonyl” is intended to indicate a radical of the formula —C(O)—NH—R, wherein R represents alkyl as defined above, e.g. methylaminocarbonyl, ethylaminocarbonyl or propylaminocarbonyl.

The term “alkylsulfonylamino” is intended to indicate a radical of the formula —NH—S(O)₂—R, wherein R represents alkyl as defined above, e.g. methylsulfonylamino.

The term “alkylsulfonylaminoalkyl” is intended to indicate a radical of the formula —R—NH—S(O)₂—R, wherein R represents alkyl as defined above, e.g. methylsulfonylaminomethyl, or methylsulfonylaminoethyl.

The term “aminosulfonylalkylaminocarbonyl” is intended to indicate a radical of the formula C(O)—NH—R—S(O)₂—NH₂, wherein R represents alkyl as defined herein, e.g. aminosulfonylmethylaminocarbonyl, or aminosulfonylethylaminocarbonyl.

The term “aminosulfonylalkyl” is intended to indicate a radical of the formula —R—S(O)₂—NH₂, wherein R represents alkyl as defined herein, e.g. aminosulfonylmethyl, aminosulfonylethyl, aminosulfonylpropyl.

The term “aminosulfonylalkylamino” is intended to indicate a radical of the formula —NH—R—S(O)₂—NH₂, wherein R represents alkyl as defined herein, e.g. aminosulfonylmethylamino, or aminosulfonylethylamino.

The term “pharmaceutically acceptable salt” is intended to indicate salts prepared by reacting a compound of formula I, Ia or Ib with a suitable inorganic or organic acid, such as hydrochloric, hydrobromic, hydroiodic, sulfuric, nitric, phosphoric, formic, acetic, 2,2-dichloroacetic, adipic, ascorbic, L-aspartic, L-glutamic, galactaric, lactic, maleic, L-malic, phthalic, citric, propionic, benzoic, glutaric, gluconic, D-glucuronic, methanesulfonic, salicylic, succinic, malonic, tartaric, benzenesulfonic, ethane-1,2-disulfonic, 2-hydroxy ethanesulfonic acid, toluenesulfonic, sulfamic or fumaric acid. Pharmaceutically acceptable salts of compounds of formula I or Ia may also be prepared by reaction with a suitable base such as sodium hydroxide, potassium hydroxide, magnesium hydroxide, calcium hydroxide, ammonia, or suitable non-toxic amines, such as lower alkylamines, for example triethylamine, hydroxy-lower alkylamines, for example 2-hydroxyethylamine, bis-(2-hydroxyethyl)-amine, cycloalkylamines, for example dicyclohexylamine, or benzylamines, for example N,N′-dibenzylethylenediamine, and dibenzylamine, or L-arginine or L-lysine.

The term “solvate” is intended to indicate a species formed by interaction between a compound, e.g. a compound of formula I, Ia or Ib and a solvent, e.g. alcohol, glycerol or water, wherein said species are in a solid form. When water is the solvent, said species is referred to as a hydrate.

Compounds of formula I, Ia or Ib may comprise asymmetrically substituted (chiral) carbon atoms and carbon-carbon double bonds which may give rise to the existence of isomeric forms, e.g. enantiomers, diastereomers and geometric isomers. The present invention includes all such isomers, either in pure form or as mixtures thereof. Pure stereoisomeric forms of the compounds and the intermediates of this invention may be obtained by the application of procedures known by persons skilled in the art. Diastereomers may be separated by physical separation methods such as selective crystallization and chromatographic techniques, e. g. liquid chromatography using chiral stationary phases. Enantiomers may be separated from each other by the selective crystallization of their diastereomeric salts with optically active acids. Alternatively, enantiomers may be separated by chromatographic techniques using chiral stationary phases. Said pure stereoisomeric forms may also be derived from the corresponding pure stereoisomeric forms of the appropriate starting materials, provided that the reaction occurs stereoselectively or stereo-specifically. Preferably, if a specific stereoisomer is desired, said compound will be synthesized by stereoselective or stereospecific methods of preparation. These methods will advantageously employ chirally pure starting materials. Likewise, pure geometric isomers may be obtained from the corresponding pure geometric isomers of the appropriate starting materials. A mixture of geometric isomers will typically exhibit different physical properties, and they may thus be separated by standard chromatographic techniques well-known in the art.

The present invention further includes prodrugs of compounds of general formula I, Ia or Ib, i.e. derivatives such as esters, ethers, complexes or other derivatives which undergo a biotransformation in vivo before exhibiting their pharmacological effects.

The compounds of formula I, Ia or Ib may be obtained in crystalline form either directly by concentration from an organic solvent or by crystallisation or re-crystallisation from an organic solvent or mixture of said solvent and a co-solvent that may be organic or inorganic, such as water. The crystals may be isolated in essentially solvent-free form or as a solvate, such as a hydrate. The invention covers all crystalline modifications and forms and also mixtures thereof.

### Embodiments

In an embodiment of the present invention compound I represent Ia or Ib

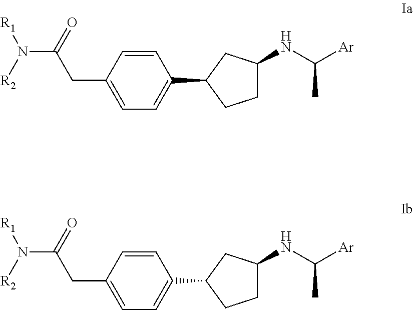

In an embodiment of the present invention Ar represents phenyl optionally substituted with one or more, same or different substituents, independently selected from chloro, fluoro or C₁₋₃alkoxy.

In an embodiment of the present invention Ar represents phenyl substituted with one or two, same or different substituents selected from chloro, fluoro or methoxy.

In an embodiment of the present invention Ar represents 4-fluoro-3-methoxyphenyl, 3-chlorophenyl, or 3-ethoxyphenyl.

In an embodiment of the present invention Ar represents C₂₋₅heterocycloalkylphenyl comprising 1-3 hetero atoms selected from O.

In an embodiment of the present invention R₁ represents C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₋₄alkyl, aminosulfonylC₁₋₄alkyl, aminocarbonylC₁₋₄alkyl, or C₃₋₅heterocycloalkyl comprising 1-2 hetero atoms selected from N, O and S, wherein said C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₋₄alkyl, aminosulfonylC₁₋₄alkyl, aminocarbonylC₁₋₄alkyl, or C₃₋₅heterocycloalkyl comprising 1-2 hetero atoms selected from N, O and S, is optionally further substituted by one or more substituents selected from halogen, hydroxy, C₁₋₂alkylsulfonylamino or —NH₂, such as methylsulfonylaminoethyl, aminosulfonylethyl, aminosulfonylpropyl, hydroxyethylaminoethyl or aminoethyl.

In an embodiment of the present invention R₂ represents hydrogen.

In an embodiment of the present invention R₁ or R₂ together with the nitrogen to which they are attached form a 4, 5, 6 or 7 membered C₃₋₆heterocycloalkyl comprising one or two heteroatoms selected from the group consisting of O, S and N, said C₃₋₆heterocycloalkyl being optionally substituted by oxo, hydroxy, trifluoromethyl, C₁₋₆alkyl, —NH₂, —S(O)₂NH₂ , —S(O)₂CH₃, C₁₋₆alkylcarbonyl, hydroxyC₂₋₆alkyl, C₁₋₆alkoxy, or C₁₋₆alkylamino, such as piperazinyl, piperidinyl, azetidinyl, or diazepanyl, optionally substituted with oxo, hydroxy, or NH₂.

Specific examples of compounds of formula I, Ia or Ib may be selected from the group consisting of

4-[2-[4-[(1R,3S)-3-[[(1R)-1-(4-fluoro-3-methoxy-phenyl)ethyl]amino]cyclopentyl]-phenyl]acetyl]piperazin-2-one; formic acid (compound 101),

2-[4-[(1R,3S)-3-[[(1R)-1-(4-fluoro-3-methoxy-phenyl)ethyl]amino]cyclopentyl]phenyl]-N-[2-(methanesulfonamido)ethyl]acetamide; formic acid (compound 102),

2-[4-[(1R,3S)-3-[[(1R)-1-(4-fluoro-3-methoxy-phenyl)ethyl]amino]cyclopentyl]phenyl]-N-(2-sulfamoylethyl)acetamide; formic acid (compound 103),

2-[4-[(1R,3S)-3-[[(1R)-1-(1,3-benzodioxol-4-yl)ethyl]amino]cyclopentyl]phenyl]-N-(2-sulfamoylethyl)acetamide (compound 104),

2-[4-[(1R,3S)-3-[[(1R)-1-(3-chlorophenyl)ethyl]amino]cyclopentyl]phenyl]-N-(2-sulfamoylethyl)acetamide (compound 105),

2-[4-[(1R,3S)-3-[[(1R)-1-(3-ethoxyphenyl)ethyl]amino]cyclopentyl]phenyl]-N-(2-sulfamoylethyl)acetamide (compound 106),

4-[2-[4-[(1R,3S)-3-[[(1R)-1-(3-ethoxyphenyl)ethyl]amino]cyclopentyl]phenyl]-acetyl]piperazin-2-one (compound 107),

2-[4-[(1R,3S)-3-[[(1R)-1-(4-fluoro-3-methoxy-phenyl)ethyl]amino]cyclopentyl]phenyl]-N-[2-(2-hydroxyethylamino)ethyl]acetamide (compound 108),

2-[4-[(1R,3S)-3-[[(1R)-1-(4-fluoro-3-methoxy-phenyl)ethyl]amino]cyclopentyl]phenyl]-1-(3-hydroxyazetidin-1-yl)ethanone (compound 109),

1-(1,4-diazepan-1-yl)-2-[4[(1R,3S)-3-[[(1R)-1-(4-fluoro-3-methoxy-phenyl)ethyl]-amino]cyclopentyl]phenyl]ethanone (compound 110),

N-(2-aminoethyl)-2-[4-[(1R,3S)-3-[[(1R)-1-(4-fluoro-3-methoxy-phenyl)ethyl]-amino]cyclopentyl]phenyl]acetamide (compound 111),

1-(4-amino-1-piperidyl)-2-[4-[(1R,3S)-3-[[(1R)-1-(4-fluoro-3-methoxy-phenyl)-ethyl]amino]cyclopentyl]phenyl]ethanone (compound 112),

2-[4-[(1R,3S)-3-[[(1R)-1-(4-fluoro-3-methoxy-phenyl)ethyl]amino]cyclopentyl]-phenyl]-1-piperazin-1-yl-ethanone (compound 113), or

2-[4-[(1R,3S)-3-[[(1R)-1-(4-fluoro-3-methoxy-phenyl)ethyl]amino]cyclopentyl]-phenyl]-N-(3-sulfamoylpropyl)acetamide (compound 114).

Specific examples of intermediates for the preparation of compounds of formula I may be selected from the group consisting of

Ethyl 2-[4-[(1R)-3-oxocyclopentyl]phenyl]acetate (Intermediate 1),

Ethyl 2-[4-[(1R,3S)-3-[[(1R)-1-(4-fluoro-3-methoxy-phenyl)ethyl]amino]-cyclo-pentyl]phenyl]acetate (Intermediate 2),

2-[4-[(1R,3S)-3-[[(1R)-1-(4-fluoro-3-methoxy-phenyl)ethyl]amino]-cyclopentyl]-phenyl]acetic acid (Intermediate 3),

(4-{(1R,3S)-3-[(1R)-1-(3-Chloro-phenyl)-ethylamino]-cyclopentyl}-phenyl)-acetic acid ethyl ester (Intermediate 4),

(4-{(1R,3S)-3-[(1R)-1-(3-Ethoxy-phenyl)-ethylamino]-cyclopentyl}-phenyl)-acetic acid ethyl ester (Intermediate 5),

{4-[(1R,3S)-3-((1R)-1-Benzo[1,3]dioxo1-4-yl-ethylamino)-cyclopentyl]-phenyl}-acetic acid ethyl ester (Intermediate 6),

(4-{(1R,3S)-3-[(1R)-1-(3-Chloro-phenyl)-ethylamino]-cyclopentyl}-phenyl)-acetic acid (Intermediate 7),

(4-{(1R,3S)-3-[(1R)-1-(3-Ethoxy-phenyl)-ethylamino]-cyclopentyl}-phenyl)-acetic acid (Intermediate 8), or

2-[4-[(1R,3S)-3-[[(1R)-1-(1,3-benzodioxol-4-yl)ethyl]amino]-cyclopentyl]-phenyl]acetic acid (Intermediate 9).

Pharmaceutical Compositions

For use in therapy, compounds of the present invention are typically in the form of a pharmaceutical composition. The invention therefore relates to a pharmaceutical composition comprising a compound of formula I, Ia or Ib, optionally together with one or more other therapeutically active compound(s), together with a pharmaceutically acceptable excipient or vehicle. The excipient must be “acceptable” in the sense of being compatible with the other ingredients of the composition and not deleterious to the recipient thereof.

Conveniently, the active ingredient comprises from 0.05-99.9% by weight of the formulation.

Pharmaceutical compositions of the invention may be in unit dosage form such as tablets, pills, capsules, powders, granules, elixirs, syrups, emulsions, ampoules, suppositories or parenteral solutions or suspensions; for oral, parenteral, opthalmic, transdermal, intra-articular, topical, pulmonal, nasal, buccal or rectal administration or in any other manner appropriate for the formulation of compounds used in nephrology and in accordance with accepted practices such as those disclosed in Remington: The Science and Practice of Pharmacy, 21^{st} ed., 2000, Lippincott Williams & Wilkins. In the composition of the invention, the active component may be present in an amount of from about 0.01 to about 99%, such as 0.1% to about 10% by weight of the composition.

For oral administration in the form of a tablet or capsule, a compound of formula I, Ia or Ib may suitably be combined with an oral, non-toxic, pharmaceutically acceptable carrier such as ethanol, glycerol, water or the like. Furthermore, suitable binders, lubricants, disintegrating agents, flavouring agents and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes or the like. Lubricants include, e.g., sodium oleate, sodium stearate, magnesium stearate, sodium benzoate, sodium acetate, sodium chloride or the like. Disintegrating agents include, e.g., starch, methyl cellulose, agar, bentonite, xanthan gum or the like. Additional excipients for capsules include macrogols or lipids.

For the preparation of solid compositions such as tablets, the active compound of formula I, Ia or Ib is mixed with one or more excipients, such as the ones described above, and other pharmaceutical diluents such as water to make a solid preformulation composition containing a homogenous mixture of a compound of formula I, Ia or Ib. The term “homogenous” is understood to mean that the compound of formula I, Ia or Ib is dispersed evenly throughout the composition so that the composition may readily be subdivided into equally effective unit dosage forms such as tablets or capsules. The preformulation composition may then be subdivided into unit dosage forms containing from about 0.05 to about 1000 mg, in particular from about 0.1 to about 500 mg, e.g. 10-200 mg, such as 30-180 mg, such as 20-50 mg of the active compound of the invention.

In the form of a dosage unit, the compound may be administered one or more times a day at appropriate intervals, always depending, however, on the condition of the patient, and in accordance with the prescription made by the medical practitioner. Conveniently, a dosage unit of a formulation contain between 0.1 mg and 1000 mg, preferably between 1 mg and 100 mg, such as 5-50 mg of a compound of formula I, Ia or Ib.

A suitable dosage of the compound of the invention will depend, inter alia, on the age and condition of the patient, the severity of the disease to be treated and other factors well known to the practising physician. The compound may be administered either orally, parenterally, intravenously or topically according to different dosing schedules, e.g. daily or with weekly intervals. In general a single dose will be in the range from 0.01 to 400 mg/kg body weight. The compound may be administered as a bolus (i.e. the entire daily dosis is administered at once) or in divided doses two or more times a day.

If the treatment involves administration of another therapeutically active compound it is recommended to consult Goodman & Gilman's The Pharmacological Basis of Therapeutics, 9^{th} Ed., J. G. Hardman and L. E. Limbird (Eds.), McGraw-Hill 1995, for useful dosages of said compounds. The administration of a compound of the present invention with one or more other active compounds may be either concomitantly or sequentially.

Liquid formulations for either oral or parenteral administration of the compound of the invention include, e.g., aqueous solutions, syrups, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrolidone.

For parenteral administration, e.g. intramuscular, intraperitoneal, subcutaneous or intravenous injection or infusion, the pharmaceutical composition preferably comprises a compound of formula I, Ia or Ib dissolved or solubilised in an appropriate, pharmaceutically acceptable solvent. For parenteral administration, the composition of the invention may include a sterile aqueous or non-aqueous solvent, in particular water, isotonic saline, isotonic glucose solution, buffer solution or other solvent conventionally used for parenteral administration of therapeutically active substances. The composition may be sterilised by, for instance, filtration through a bacteria-retaining filter, addition of a sterilising agent to the composition, irradiation of the composition, or heating the composition. Alternatively, the compound of the invention may be provided as a sterile, solid preparation, e.g. a freeze-dried powder, which is dissolved in sterile solvent immediately prior to use.

The composition intended for parenteral administration may additionally comprise conventional additives such as stabilisers, buffers or preservatives, e.g. antioxidants such as methyl hydroxybenzoate or the like.

Compositions for rectal administration may be in the form of a suppository incorporating the active ingredient and a carrier such as cocoa butter, or in the form of an enema.

Compositions suitable for intra-articular administration may be in the form of a sterile aqueous preparation of the active ingredient which may be in microcrystalline form, for example, in the form of an aqueous microcrystalline suspension. Liposomal formulations or biodegradable polymer systems may also be used to present the active ingredient for both intra-articular and ophthalmic administration.

Compositions suitable for topical administration, including ophthalmic treatment, include liquid or semi-liquid preparations such as liniments, lotions, gels, applicants, oil-in-water or water-in-oil emulsions such as creams, ointments or pastes; or solutions or suspensions such as drops. For topical administration, the compound of formula I, Ia or Ib may typically be present in an amount of from 0.01 to 20% by weight of the composition, such as 0.1% to about 10%, but may also be present in an amount of up to about 50% of the composition. Compositions for ophthalmic treatment may preferably additionally contain a cyclodextrin. Compositions suitable for administration to the nasal or buccal cavity or for inhalation include powder, self-propelling and spray formulations, such as aerosols and atomizers. Such compositions may comprise a compound of formula I, Ia or Ib in an amount of 0.01-20%, e.g. 2%, by weight of the composition.

The composition may additionally comprise one or more other active components conventionally used in the treatment of physiological disorders or diseases associated with disturbances of CaSR activity, such as hyperparathyroidism.

Pharmacological Methods

The calcium sensing receptor (CaSR) and its use in identifying or screening for calcimimetic compounds has e.g. been described in EP 637 237, EP 1 296 142, EP 1 100 826, EP 1 335 978, and EP 1 594 446.

In vitro and in vivo methods for testing the compounds of the present invention are well established and may be found in the references listed above, or e.g. in Journal of Biological Chemistry (2004), 279(8), 7254-7263 or in U.S. Pat. No. 5 858 684 and references cited therein.

Biological Assay for Analysis of In Vitro Activity

The assay investigates a compound's functional ability to act as a biological positive modulator on the human CaSR. Activation of the receptor expressed on CHO-K1 cells is detected through the G alpha q pathway, the activation of phospholipase C and the accumulation of intracellular inositol phosphate (IP) as described earlier [Sandrine Ferry, Bruno Chatel, Robert H. Dodd, Christine Lair, Danielle Gully, Jean-Pierre Maffrand, and Martial Ruat. Effects of Divalent Cations and of a Calcimimetic on Adrenocorticotropic Hormone Release in Pituitary Tumor Cells. Biological and Biophysical Research Comm., 238, 866-873 (1997)]. The human CaSR is stably expressed on a CHO-K1 cell clone, stimulated with a basal level of calcium and challenged with the tested compound. The level of IP1 is determined using the IP-One Terbium htrf kit (Cisbio, France). CHO-K1 cells not transfected with the CaSR fail to elicit an IP1 response upon calcium and/or compound stimulation.

Cloning of the Human CaSR Gene

The ORF coding for the human CaSR (genebank: NM_{—}000388) was acquired from Invitrogen Corp, USA and subsequently cloned into the mammalian expression vector pCDA3.1.

Generation of Cell Line Expressing CaSR

CHO-K1 cells were transfected using Lipofectamine according to manufacturer's protocol (400.000 cells/well were seeded in a 6-well plate and transfected after 24 hours using 2 μg DNA and 5 μl lipofectamine). After another 24 hours the cells were detached, seeded and subjected to 1mg/ml of G-418. Following 7 days growth single clones were picked, the CaSR expression evaluated using the 5C10 antibody against CaSR, the clones with the highest expression were selected and tested for functional response. The preferred clone was cultured according to standard procedures described in ATCC (American Type Culture Collection) protocols for CHO-K1 with the addition of 500 μg/ml G-418.

Functional Whole Cell Assay

On the assay day, cells were thawed, harvested and resuspended to 4*10⁶ cells/ml in stimulation buffer (containing: Hepes 10mM, MgCl₂ 0.5mM, KCl 4.2 mM, NaCl 146 mM, glucose 5.5 mM, LiCl 50 mM, BSA 0.5% at pH 7.4). Ten μl cell solution was pipetted into wells of a white 384-well plate (Perkin Elmer Optiplate) containing 2 μl compound diluted in assay buffer (containing: Hepes 10 mM, MgCl₂ 0.5mM, KCl 4.2mM, NaCl 146mM, glucose 5.5 mM, LiCl 50 mM, CaCl₂ 11.4 mM at pH 7.4), resulting in a final Ca²⁺ concentration of 1.9 mM. After compound stimulation for 1 hour at 37° C. and 15 min at room temperature, 10 ul of IP-One assay detection reagent (prepared as described by the IP-One assay kit manufacturer) was added and the plate was incubated for another 1 hour at room temperature. Finally the plate was read using a Perkin Elmer EnVision, according to the protocol supplied by the IP-One assay kit manufacturer. The FRET ratio was calculated by dividing the 665 nm emission signal with that of the 615 nm.

The molar concentration of a compound that produces 50% of the maximum agonistic response (the IC50 value) is calculated according to the equation “General sigmoidal curve with Hill slope, a to d” (Equation 1). This model describes a sigmoidal curve with an adjustable baseline. The equation can be used to fit curves where response is either increasing or decreasing with respect to the independent variable, X.

y=(a−d)/(1+(x/c)̂b)+d Equation 1.

Parameters:

x=concentration of tested compound

y=response (%)

a=min response as compound concentration approaches 0

d=max response as concentration of tested compound is increasing

c=IC50 for the curve

b=Hill coefficient or curve slope

Assay results using compounds of the present invention indicate that compounds of the present invention are potent modulators of CaSR, thus making them potentially useful in the treatment of diseases related to kidneys or bones. See table 1.

Biological assay for analysis of clearance in human liver microsomes Test compound concentration is 0.5 μM, microsome concentration is 0.5 mg/mL and NADPH concentration is 1 mM in the incubation. The described method is performed by the liquid handling system Tecan RSP and is based on a 96-well format.

Control incubations with test compound without NADPH and test compound without microsomes are conducted to investigate non-CYP mediated metabolism and stability in phosphate buffer at 37° C., respectively.

Incubation Conditions

The human liver microsomal suspension in phosphate buffer is mixed with NADPH. The mixture is pre-heated (7 min) to 37° C. Test compound is added, and the mixture is incubated for 30 minutes. Incubations are run in duplicate. Samples are withdrawn at predetermined stop times and mixed with methanol containing internal standard (IS) to terminate all enzyme activity and precipitate proteins. A control without NADPH (to detect problems such as nonspecific protein binding, heat instability or non-CYP mediated metabolism) and a control without microsomes (for assessing compound stability in the absence of any active enzymes) are tested.

The percentage of organic solvent in the incubations is less than 1%. Careful inspections of reagents are performed prior to the start of any experiment to ensure all reagents are in solution.

Sample Analysis

The 96-well plates are centrifuged. Test compound depletion, using a compound specific LC/MS/MS method, is determined.

The logarithm of the peak area ratios of test compound to internal standard (IS) versus incubation time is plotted in a graph.

The rate constant (k) (min⁻¹) of test compound depletion is calculated from the linear part of the curve and the half-time (t_{1/2}) in minutes can be calculated from the rate constant (Eq. 2).

t_{1/2}=(ln2)/k (Eq. 2)

Intrinsic clearance (Clᵢₙₜ) (mL/min/mg protein) is calculated from:

Clᵢₙₜ=k/c (Eq. 3)

where c is the microsomal protein concentration in mg/mL.

Intrinsic clearance is the maximum ability of the liver to extract a drug in the absence of blood flow restrictions.

Conversion to apparent clearance (Clₐₚₚ) (mL/min/kg) is done by Eq. 4:

Clₐₚₚ=Clᵢₙₜ×a×b/d (Eq. 4)

where a, b and d are the scaling factors for normalizing Clᵢₙₜ to human body weight.

The following human scaling factors are used:

a: 45 (microsomal protein/liver weight (mg/g))

b: 1500 (liver weight (g))

d: 70 (body weight (kg))

Hepatic clearance (Clₕ) (mL/min/kg) based on the well-stirred model is described as followed:

Clₕ=(Clₐₚₚ·Q)/(Clₐₚₚ+Q) (Eq. 5)

where Q is the liver blood flow in mL/min/kg (20 in humans).

Dividing hepatic clearance with liver blood flow, the hepatic extraction ratio (%) can be calculated:

Eₕ=Clₕ/Q·100 (Eq. 6)

Apparent clearance below approximately 10 mL/min/kg human body weight (corresponding to extraction ratio of approx. 33%) is considered as low clearance (high metabolic stability). Apparent intrinsic clearance above approximately 60 mL/min/kg human body weight (corresponding to extraction ratio of approx. 75%) is considered as high clearance (low metabolic stability).

Results for compounds according to the present invention tested in the above assay are shown in table 1.

Biological Assay for Analysis of Clearance in Rat Hepatocytes

Test compounds and 4 control compounds are tested in duplicate per run. Test compound concentration is 0.5 μM and cell concentration is 1×10⁶ cells/mL in the incubation.

The described method is performed by the liquid handling system Tecan RSP and is based on a 96-well format.

The liver is collected from a male Spraque-Dawley rat. One liver lobe is cut off and flushed with various buffers to loosen the cells. The cell suspension is washed and centrifuged, and the cell density is adjusted to 1.2×10⁶ cells/mL with Krebs-Henseleit buffer, pH 7.4, containing 0.2% bovine serum albumin (BSA). Only cell suspensions with viability above 80% are used.

Incubation Conditions

The cell suspension is pre-heated (20 min) to 37° C. Test compound is added, and the mixture is incubated for 20 minutes. Incubations are run in duplicate. Samples are withdrawn at predetermined stop times and mixed with methanol containing internal standard (IS) to terminate all enzyme activity and precipitate proteins.

The percentage of organic solvent in the incubations is less than 1%. Careful inspections of reagents are performed prior to the start of any experiment to ensure all reagents are in solution.

Sample Analysis

The 96-well plates are centrifuged. Test compound depletion, using a compound specific LC/MS/MS method, is determined.

Data Analysis

Data analysis is performed as described above in the section “Biological assay for analysis of clearance in human liver microsomes”, with the following modifications:

Intrinsic clearance (Clᵢₙₜ) (mL/min/10⁶ cells) is calculated from:

Clᵢₙₜ=k/c

where c is the cell concentration in 10⁶ cells/mL.

The following scaling factors for rat are used for eq. 4:

a: 120 (cells/liver weight (10⁶ cells/g))

b: 10 (liver weight (g))

d: 0.25 (body weight (kg))

Liver blood flow in rats (for eq. 5):

Q: 55 mL/min/kg

Apparent clearance below approximately 25 mL/min/kg rat body weight (corresponding to an extraction ratio of approx. 33%) is considered as low clearance (high metabolic stability). Apparent intrinsic clearance above approximately 165 mL/min/kg rat body weight (corresponding to an extraction ratio of approx. 75%) is considered as high clearance (low metabolic stability).

Results for compounds according to the present invention tested in the above assay are shown in table 1.

TABLE 1
Pharmacokinetic data for compounds according to the present invention.

| | | Clearance (% Eh) in | |
|---|---|---|---|
| | Functional whole | human liver | Clearance (% Eh) in |
| | cell assay | microsomes | rat hepatocytes |
| | (modulation of | A: Eh < 33%; | A: Eh < 33%; |
| | human CaSR) | B: 33% ≦ Eh ≦ | B: 33% ≦ Eh ≦ |
| Compound | A: <200 nM; | 75%; | 75%; |
| number | B: 200-1000 nM; | C: Eh > 75% | C: Eh > 75% |
| 102 | B | A | — |
| 103 | A | A | B |
| 104 | A | A | A |
| 106 | A | A | B |
| 108 | A | A | B |
| 109 | A | A | B |
| 110 | A | B | B |
| 112 | A | A | B |

Methods of Preparation

The compounds of general formula I can be prepared in a number of ways well known to those skilled in the art of organic synthesis. The compounds of formula I can be synthesized using the methods outlined below, together with methods known in the art of synthetic organic chemistry, or variations thereof as appreciated by those skilled in the art. Preferred methods include, but are not limited to, those described below.

The compounds of formula I can be prepared by techniques and procedures readily available to one of ordinary skill in the art, for example by following the procedures as set forth in the following schemes. The reactions are performed in solvents appropriate to the reagents and materials employed and suitable for the transformations being effected. Also, in the synthetic methods described below, it is to be understood that all proposed reaction conditions, including choice of solvent, reaction atmosphere, reaction temperature, duration of experiment and work-up procedures, are chosen to be conditions of standard for that reaction, which should be readily recognised by one skilled in the art. It is understood by one skilled in the art of organic synthesis that the functionalities present on various portions of the starting molecules in a reaction must be compatible with the reagents and reactions proposed. Not all compounds of formula I falling into a given class may be compatible with some of the reaction conditions required in some of the methods described. Such restrictions to the substituents which are compatible with the reaction conditions will be readily apparent to one skilled in the art and alternative methods can be used.

The schemes described in this section are not intended to limit the scope of the invention in any way. All substituents, unless otherwise indicated, are previously defined. The reagents and starting materials are either available from commercial suppliers or prepared by methods known to one of ordinary skill in the art following procedures set forth in references such as Fieser and Fieser's Reagents for Organic Synthesis, Volumes 1-22 (John Wiley and Sons, 2004); Rodd's Chemistry of Carbon Compounds, Volumes 1-5 and Supplements (Elsevier Science Publishers, 2000); Organic Reactions, Volumes 1-64 (John Wiley and Sons, 2004); March's Advanced Organic Chemistry (John Wiley and Sons, 5^{th} Edition) and Larock's Comprehensive Organic Transformations (VCH Publishers Inc., 1999). These schemes are merely illustrative of some methods by which the compounds of this invention can be synthesised, and various modifications to these schemes can be made and will be suggested to one skilled in the art having referred to this disclosure. The starting materials and the intermediates of the reactions may be isolated and purified if desired using conventional techniques, including but not limited to filtration, distillation, crystallisation, chromatography and the like. Such materials may be characterised using conventional means, including physical constants and spectral data.

Compounds of general formula I may be obtained by reductive amination between a cyclopentanone of general formula II and an amine of general formula III. The reaction between ketone II and amine III may be carried out either by one-pot reductive amination or with isolation of the imine followed by reduction.

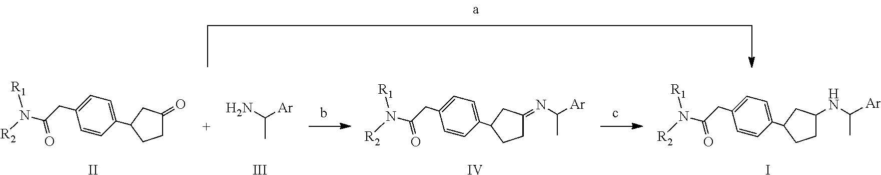

a. The formation of the intermediate iminium IV may be promoted by addition of a protic or aprotic acid such as, but not limited to, acetic acid, Yb(OAc)₃ and Ti(Oi-Pr)₄. The reducing agent may be but is not limited to Na(CN)BH₃, NaBH₄, Na(OAc)₃BH (for other non-limiting conditions see Org. React. 2002, 59, 1-714 and references cited therein).

b. The formation of the imine is promoted either by Lewis acids such as TiCl₄, ZnCl₂, AlCl₃ or by bases such as pyridine, optionally in the presence of a drying agent such as TiCl₄ or molecular sieve (see Comprehensive Organic Functionnal Group Transformations 3, 403 (1995) Pergamon).

c. Reduction may be performed by hydrogenation in the presence of a catalyst such as Pd/C, Pt/C or a chiral rhodium complex to perform the reaction in a stereoselective manner or by hydride transfer from a reducing agent such as BH₃, NaBH₄, NaBH₃CN, LiAlH₄, L-selectride (see Larock R. C. Comprehensive Organic Transformations 1989, VCH; Comprehensive Organic Functional Group Transformations 2, 268-269 (2005) Pergamon and references cited therein).

The amide II may be prepared from the carboxylic acid VI by standard amide coupling with an amine R₁R₂NH. Standard amide coupling may involve the activation of the carboxylic acid using reagents such as EDAC, DIC, DCC, CDI, PyBOP, HOBt, HATU or HOAt in solvents such as DMF, THF, DCM, MeCN or H₂O or mixtures thereof, optionally in the presence of a base such as Et₃N or DIPEA.

The carboxylic acid VI may in turn be prepared from the corresponding alkyl ester V (wherein R8=alkyl) by hydrolysis using a base such as NaOH, LiOH or KOH or a mineral acid such as HCl or H₂SO₄ in solvents such as MeOH, EtOH, or H₂O or mixtures thereof.

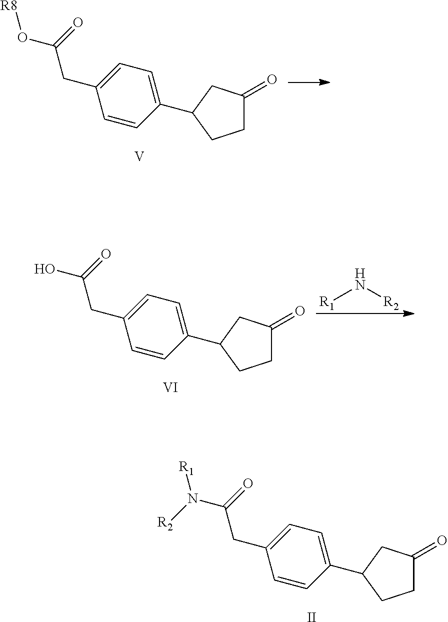

The cyclopentanone V may be prepared from 2-cyclopentenones:

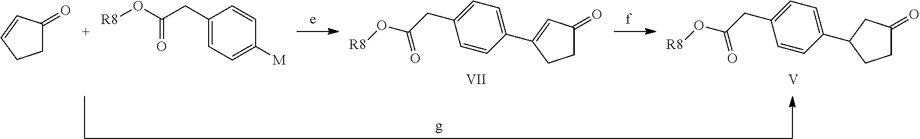

e. Coupling reaction with an arylhalide or pseudo halide such as triflate in the presence of a palladium source such as Pd(OAc)₂, PdCl₂(PPh₃)₂, a base such as NEt₃, K₂CO₃, NaHCO₃, optionally with a phosphine such PPh₃, P(o-Tol)₃, 1,3-bis(diphenylphosphino)propane (dppp), optionally in the presence of a salt like NBu₄Cl, AgNO₃ in a solvent such as DMF or acetonitrile. Alternatively a decarboxylative Heck-type coupling may be performed using an aryl carboxylic acid (Org. Lett. 2004, 6, 433).

f. Chemospecific reduction of the double bond may be performed under numerous conditions. The hydrogen source may be H₂, water, Hantzsch esters. Metal-based catalysts such as Pd/C, Pd(PPh₃)₄, supported PdCl₂, Rh—, Co—, Cu—, Ir-based catalysts may be used. Stereoselectivity may be achieved by addition of a chiral auxiliary such as but not limited to enantiopure binaphtol phosphate derivatives/valine, imidazolidinone iminiums, bidentate phosphines.

Alternatively cyclopentenones may be subjected to 1,4-addition. g. Reaction with an arylmetal in which the metal may be Li, Mg halide, trialkyltin, boronic acid, boronic acid ester, optionally in the presence of a metal complex such as PdCl₂, Pd(OAc)₂, Pd(PPh₃)₄, (acac)Rh(CO)₂, Ni(acac)₂, (COD)Rh(1,4-dihydroquinone)BF₄ with a ligand typically phosphine-based such as PBu₃, PPh₃, 1,3-bis(diphenylphosphino)propane (dppp), 1,3-hydroquinone or 1,4-hydroquinone in solvents such as DMF, THF, water, toluene, dioxane, dimethoxyethane. In the presence of a chiral ligand as a pure enantiomer such as BINAP, phosphoramidite, Me-DuPHOS and the like the reaction may be performed stereoselectively.

Compounds of general formula I may also be prepared from cyclopentanone V in the following manner:

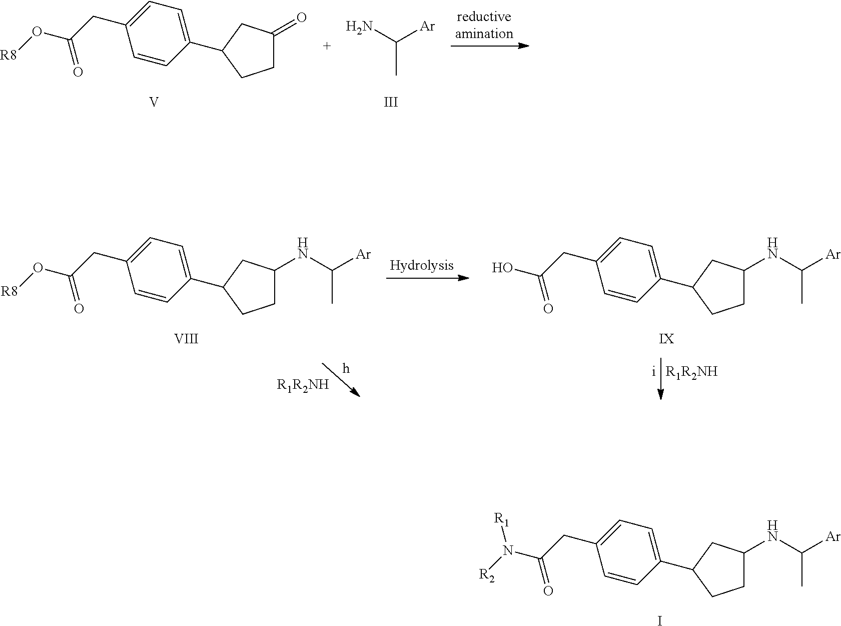

Reductive amination between V and III is carried out as described above for the reductive amination between II and III.

h. The alkyl ester VIII thus formed may be converted directly to amides of the general formula I by reaction with an amine R₁R₂NH. Such a reaction may be carried out in a solvent such as, but not limited to, MeOH, EtOH, DCM, H₂O, THF, DMF, or dioxane and with optional heating.

Alternatively, the alkyl ester VIII may be hydrolysed to the carboxylic acid IX, which in turn may be converted to the amide I by coupling with an amine. The hydrolysis may be carried out as described above for the conversion of V to VI. The amide formation may be carried out as described above for the conversion of VI to II.

Chiral amines of the general formula III are commercially available or may be prepared from more readily available aldehydes by catalytic asymmetric synthesis using tert-butanesulfinamide according to Liu, G.; Cogan, D. A.; Ellmann, J. A., J. Amer. Chem. Soc., 1997, 114, 9913.

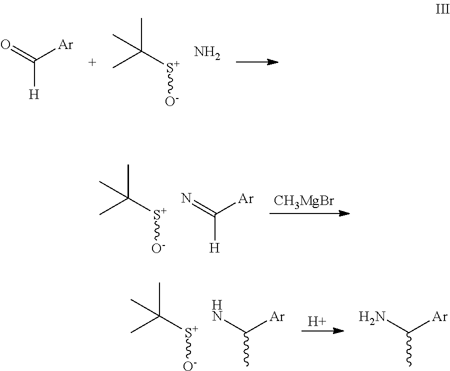

Diastereomeric mixtures of I, VIII, and IX may be separated using straight phase chromatography on silica gel, by preparative HPLC or by chiral HPLC.

The invention is described in further detail in the following non-limiting examples which are not in any way intended to limit the scope of the invention as claimed.

### EXAMPLES

General

For ¹H nuclear magnetic resonance (NMR) spectra (300 MHz) and ¹³C NMR (75.6 MHz) chemical shift values (δ) (in ppm) are quoted for dimethyl-d₆ sulfoxide (DMSO-d₆) or CDCl₃ solutions relative to internal tetramethylsilane (δ=0) standard. The value of a multiplet, either defined (doublet (d), triplet (t), quartet (q), doublet of doublets (dd), doublet of triplets (dt)) or not (m) at the approximate mid point is given unless a range is quoted, (bs) indicates a broad singlet. The ES mass spectra were obtained on a VG Quattro II triple quadrapole mass spectrometer (Micromass, Manchester, UK) operating in either positive or negative electrospray mode with a cone voltage of 30V.

The microwave reactor used was the model Initiator™ from Biotage. The organic solvents used were anhydrous unless otherwise specified. Flash chromatography was performed on silica gel from Fluka Chemie GmbH, Switzerland.

Chemicals unless otherwise noted were from commercial sources, e.g. Aldrich, Maybridge Chemical, Fluka or ABCR.

Abbreviations
Acac Acetyl acetonate
BOC tert-Butyl oxycarbonyl
CDI N,N′-Carbonyl diimidazole
COD 1,5-Cyclooctadiene
DCC N,N′-dicyclohexylcarbodiimide
DCM Dichloromethane
DIC Di-isopropyl carbodiimide
DIPEA Diisopropyl ethylamine
DMF N,N-Dimethyl formamide
DMSO Dimethylsulfoxide
EDAC N-Ethyl N′-(3-dimethylaminopropyl)carbodiimide hydrochloride
HATU (2-(7-Aza-1H-benzotriazole-1-yl)-1,1,3,3-tetramethyluronium hexafluorophosphate)
HOAt 1-Hydroxy-7-Azabenzotriazole
HOBt 1-Hydroxy-benzotriazole
NMP N-methylpyrrolidinone
P(o-Tol)₃ tri-o-tolylphosphine
PyBOP benzotriazol-1-yl-oxytripyrrolidinophosphonium hexafluorophosphate
RT Retention time
rt Room temperature

Flash chromatography was performed on silica gel. Appropriate mixtures of ethyl acetate, dichloromethane, methanol, and heptane were used as eluents unless otherwise noted.

[Rh(R-BINAP)(nbd)]BF₄ was prepared according to the procedure described in Itooka, R.; Iguchi, Y.; Miyaura, N.; J. Org. Chem., 2003, 68, 6000.

HPLC purifications of the crude products were performed by using Waters LC-MS system [column: Waters X Terra C18, 5 μm or Luna C18 100 Å 5μ; Size: 250×10.00 mm (Phenomenex)]; Sample Manager: Waters 2767; Pump: Waters 2525; Single Quadrupole: Waters ZQ; PDA-detector: Waters 2996), solventsystem: A=50 mM Ammonium hydrogencarbonate and B=acetonitrile; flow rate=18 mL/min.

TABLE 2
Exemplified compounds of general formula I:

| Compound | Structure |
|---|---|
| 101 | 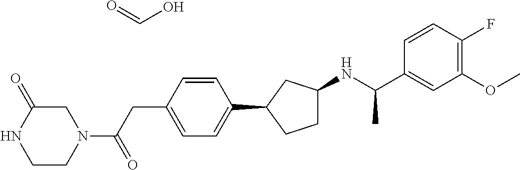 [SMILES: O=CO ; COc1cc([C@@H](C)N[C@H]2CC[C@@H](c3ccc(CC(=O)N4CCNC(=O)C4)cc3)C2)ccc1F] |
| 102 | 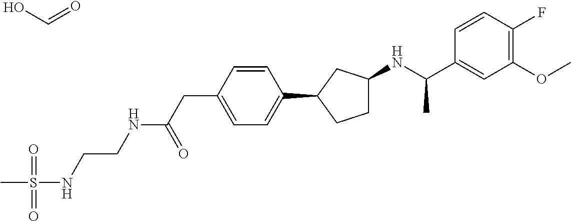 |
| 103 | 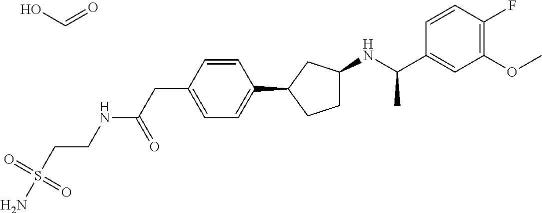 [SMILES: COc1cc([C@@H](C)N[C@H]2CC[C@@H](c3ccc(CC(=O)NCCS(N)(=O)=O)cc3)C2)ccc1F ; O=CO] |
| 104 | 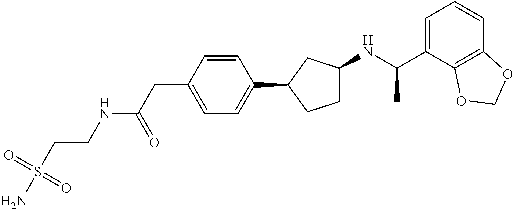 [SMILES: C[C@@H](N[C@H]1CC[C@@H](c2ccc(CC(=O)NCCS(N)(=O)=O)cc2)C1)c1cccc2c1OCO2] |
| 105 | 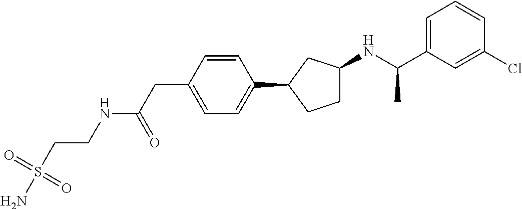 [SMILES: C[C@@H](N[C@H]1CC[C@@H](c2ccc(CC(=O)NCCS(N)(=O)=O)cc2)C1)c1cccc(Cl)c1] |
| 106 | 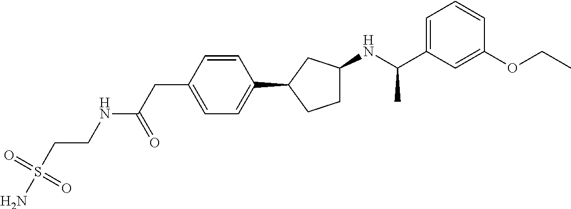 [SMILES: CCOc1cccc([C@@H](C)N[C@H]2CC[C@@H](c3ccc(CC(=O)NCCS(N)(=O)=O)cc3)C2)c1] |
| 107 | 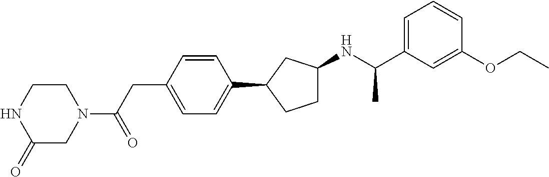 [SMILES: CCOc1cccc([C@@H](C)N[C@H]2CC[C@@H](c3ccc(CC(=O)N4CCNC(=O)C4)cc3)C2)c1] |
| 108 | 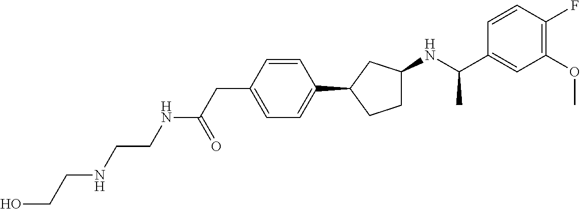 [SMILES: COc1cc([C@@H](C)N[C@H]2CC[C@@H](c3ccc(CC(=O)NCCNCCO)cc3)C2)ccc1F] |
| 109 | 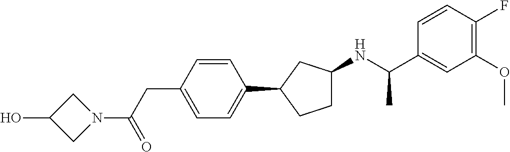 [SMILES: COc1cc([C@@H](C)N[C@H]2CC[C@@H](c3ccc(CC(=O)N4CC(O)C4)cc3)C2)ccc1F] |
| 110 | 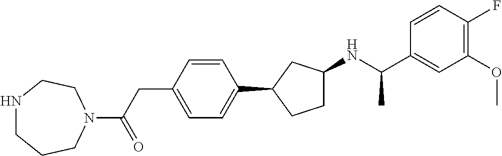 [SMILES: COc1cc([C@@H](C)N[C@H]2CC[C@@H](c3ccc(CC(=O)N4CCCNCC4)cc3)C2)ccc1F] |
| 111 | 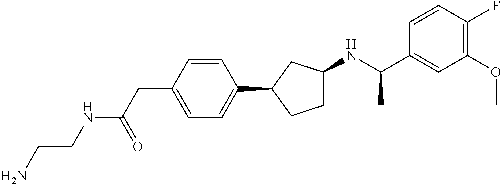 [SMILES: COc1cc([C@@H](C)N[C@H]2CC[C@@H](c3ccc(CC(=O)NCCN)cc3)C2)ccc1F] |
| 112 | 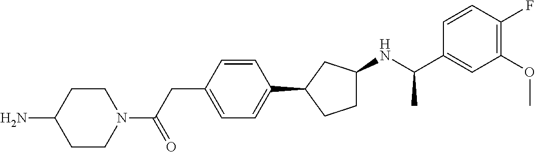 [SMILES: COc1cc([C@@H](C)N[C@H]2CC[C@@H](c3ccc(CC(=O)N4CCC(N)CC4)cc3)C2)ccc1F] |
| 113 | 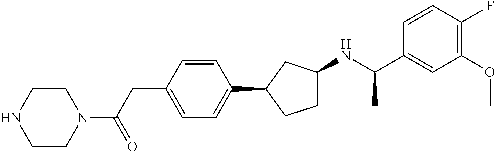 [SMILES: COc1cc([C@@H](C)N[C@H]2CC[C@@H](c3ccc(CC(=O)N4CCNCC4)cc3)C2)ccc1F] |
| 114 | 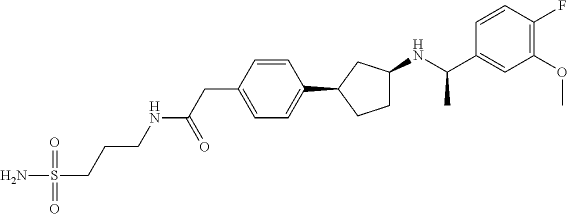 [SMILES: COc1cc([C@@H](C)N[C@H]2CC[C@@H](c3ccc(CC(=O)NCCCS(N)(=O)=O)cc3)C2)ccc1F] |

TABLE 3
Exemplified intermediates:

| Intermediate | Structure |
|---|---|
| 1 | 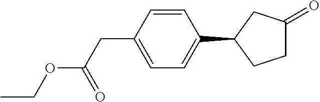 [SMILES: CCOC(=O)Cc1ccc([C@@H]2CCC(=O)C2)cc1] |
| 2 | 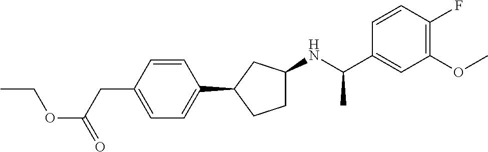 [SMILES: CCOC(=O)Cc1ccc([C@@H]2CC[C@H](N[C@H](C)c3ccc(F)c(OC)c3)C2)cc1] |
| 3 | 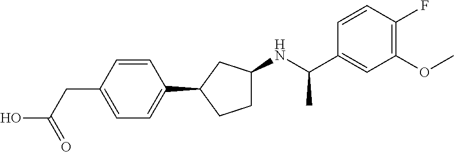 [SMILES: COc1cc([C@@H](C)N[C@H]2CC[C@@H](c3ccc(CC(=O)O)cc3)C2)ccc1F] |
| 4 | 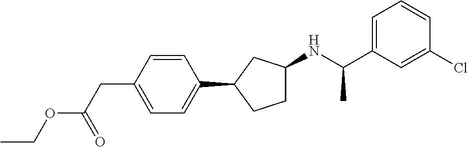 [SMILES: CCOC(=O)Cc1ccc([C@@H]2CC[C@H](N[C@H](C)c3cccc(Cl)c3)C2)cc1] |
| 5 | 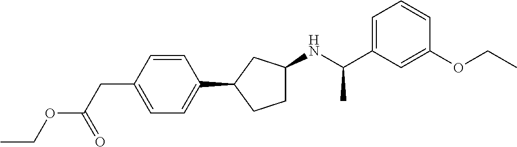 [SMILES: CCOC(=O)Cc1ccc([C@@H]2CC[C@H](N[C@H](C)c3cccc(OCC)c3)C2)cc1] |
| 6 | 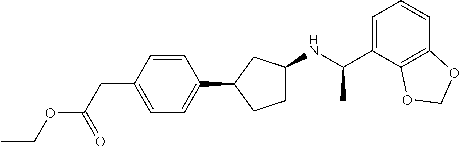 [SMILES: CCOC(=O)Cc1ccc([C@@H]2CC[C@H](N[C@H](C)c3cccc4c3OCO4)C2)cc1] |
| 7 | 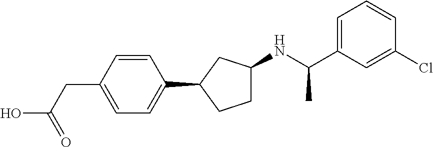 [SMILES: C[C@@H](N[C@H]1CC[C@@H](c2ccc(CC(=O)O)cc2)C1)c1cccc(Cl)c1] |
| 8 | 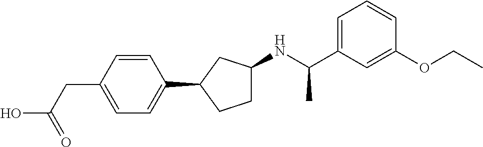 [SMILES: CCOc1cccc([C@@H](C)N[C@H]2CC[C@@H](c3ccc(CC(=O)O)cc3)C2)c1] |
| 9 | 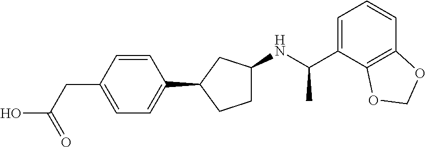 [SMILES: C[C@@H](N[C@H]1CC[C@@H](c2ccc(CC(=O)O)cc2)C1)c1cccc2c1OCO2] |

General Procedure A (Amide Formation)

An acid (0.05 mmol) was dissolved/suspended in 0.25 mL of NMP and treated with 1.1 eq. of CDI for 1 h at rt. The activated acid was added to an amine (0.1 mmol) and DIPEA (0.15 mmol), and the mixture was left overnight at rt. The progress of the reaction was monitored by LCMS, and if more than 50% of the acid remained, additional amine (1 eq) and EDAC (1 eq.) was added, and the mixture was allowed to stand another 3 h. The product was purified by preparative HPLC. If a BOC-protected amine was used, the BOC-protected intermediate was deprotected prior to purification: The intermediate was extracted with EtOAc, concentrated in vacuo, treated with TFA for 1 h, concentrated in vacuo, and redissolved in NMP and a few drops of H₂O.

General Procedure B (Amide Formation)

A solution of acid (0.030 mmol) in 0.2 mL DMF was treated with CDI (0.036 mmol) for 20 min at rt. DIPEA (0.089 mmol) and an amine (0.089 mmol) were added, and the mixture was stirred overnight at rt. If unreacted acid remained, DIC (5 μL) was added, and the mixture was stirred another 4 h at rt. The product was purified directly by preparative HPLC (10-100% CH₃CN in H₂O/0.1% HCOOH).

General Procedure C (Amide Formation)

A solution of acid (0.060 mmol) in 0.3 mL DMF was treated with HOBt (0.06 mmol), DIPEA (0.18 mmol) and EDAC (0.066 mmol) for 10 min at rt and subsequently added to an amine (0.12 mmol). The mixture was allowed to stand for 1 h at rt. The product was purified directly by preparative HPLC.

### Intermediate 1: Ethyl 2-[4-[(1R)-3-oxocyclopentyl]phenyl]acetate

[Rh(R-BINAP)(nbd)]BF₄ (0.22 mmol) and (4-ethoxycarbonylmethylphenyl)boronic acid pinacol ester (8.6 mmol) were dissolved in 5 mL MeOH in a sealed 20 mL MW-vial. The flask was flushed with argon. A solution of triethylamine (8.6 mmol) and 2-cyclopenten-1-one (21.5 mmol) in MeOH (5 mL), degassed with argon, was then added. The mixture was heated for 20 min at 100° C. in a microwave reactor. The reaction mixture was diluted with 40 mL DCM, washed twice with sat. NaHCO₃ and H₂O, dried over MgSO₄, filtered, and concentrated in vacuo affording a dark oil. Purification by flash chromatography (gradient of 0-50% EtOAc in heptane) afforded the title compound as a pale yellowish oil.

¹H NMR (300 MHz, CDCl₃) δ 7.29-7.18 (m, 4H), 4.15 (q, J=7.2 Hz, 2H), 3.60 (s, 2H), 3.47-3.33 (m, 1H), 2.72-2.60 (m, 1H), 2.52-2.22 (m, 4H), 2.05-1.90 (m, 1H), 1.26 (d, J=7.1 Hz, 3H).

### Intermediate 2: Ethyl 2-[4-[(1R,3S)-3-[[(1R)-1-(4-fluoro-3-methoxy-phenyl)ethyl]-amino]-cyclopentyl]phenyl]acetate

A solution of Intermediate 1 (3.25 mmol) in acetonitrile (4 mL) was treated with (1R)-1-(4-fluoro-3-methoxyphenyl)ethylamine hydrochloride (3.57 mmol) and NaBH(OAc)₃ (4.87 mmol) for 2 h at rt. The reaction mixture was diluted with additional acetonitrile (10 mL) and loaded on silica gel. The two diastereomers were separated by flash chromatography (gradient of 0-50% of EtOAc in n-heptane). The title compound was obtained in 31% yield as the faster eluting isomer.

¹H NMR (300 MHz, CDCl₃) δ 7.23-7.10 (m, 4H), 7.01 (dd, J=11.1, 8.2 Hz, 2H), 6.82 (ddd, J=8.1, 4.3, 1.9 Hz, 1H), 4.14 (q, J=7.2 Hz, 2H), 3.91 (s, 3H), 3.85 (q, J=6.1 Hz, 1H), 3.56 (s, 2H), 3.13-3.00 (m, 1H), 2.99-2.84 (m, 1H), 2.26-2.13 (m, 1H), 2.08-1.89 (m, 2H), 1.85-1.30 (m, 7H), 1.24 (d, J=7.1 Hz, 3H).

### Intermediate 3: 2-[4-[(1R,3S)-3-[[(1R)-1-(4-Fluoro-3-methoxy-phenyl)ethyl]amino]-cyclopentyl]phenyl]acetic acid

Intermediate 2 (0.75 mmol) was dissolved in 6 mL of THF-MeOH (1:1) and treated with 4M NaOH (0.5 mL) and water (0.5 mL) for 1 h at rt. After concentrating the mixture in vacuo, the pH was adjusted to approx. 5 with 1M HCl and extracted with DCM. The combined organic extracts were dried, filtered and concentrated in vacuo affording the title compound in quantitative yield as a colorless foam.

¹H NMR (600 MHz, DMSO) δ 7.25 (dd, J=8.4, 1.5 Hz, 1H), 7.18-7.10 (m, 5H), 6.94 (ddd, J=8.0, 4.1, 1.8 Hz, 1H), 3.90 (q, J=6.6 Hz, 1H), 3.83 (s, 3H), 3.46 (s, 2H), 3.00-2.93 (m, 1H), 2.89-2.81 (m, 1H), 2.11-2.04 (m, 1H), 1.90-1.77 (m, 2H), 1.72-1.59 (m, 2H), 1.43 (td, J=11.8, 9.5 Hz, 1H), 1.32 (d, J=6.6 Hz, 3H).

### Intermediate 4: (4-{(1R,3S)-3-[(1R)-1-(3-Chloro-phenyl)-ethylamino]-cyclo-pentyl}-phenyl)-acetic acid ethyl ester

A solution of Intermediate 1 (0.81 mmol) in acetonitrile (4 mL) was treated with (R)-1-(3-chlorophenyl)ethanamine (0.89 mmol) and NaBH(OAc)₃ (1.22 mmol) at rt overnight. The reaction mixture was diluted with additional acetonitrile (5 mL) and loaded on silica gel. The two diastereomers were separated by flash chromatography (gradient of 0-30% of EtOAc/2% Et₃N/1% i-PrOH in n-heptane:Et₃N (100:2)). The title compound was obtained in 24% yield as the faster eluting isomer.

¹H NMR (300 MHz, CDCl₃) δ 7.32 (d, J=1.7 Hz, 1H), 7.28-7.10 (m, 7H), 4.13 (q, J=7.1 Hz, 2H), 3.83 (q, J=6.7 Hz, 1H), 3.56 (s, 2H), 3.10-2.83 (m, 2H), 2.23-2.12 (m, 1H), 2.07-1.88 (m, 2H), 1.83-1.30 (m, 7H), 1.24 (t, J=7.1 Hz, 3H).

### Intermediate 5: (4-{(1R,3S)-3-1(1R)-1-(3-Ethoxy-phenyl)-ethylamino]-cyclo-pentyl}-phenyl)-acetic acid ethyl ester

The procedure described for intermediate 4 was followed, using Intermediate 1 as the ketone and (R)-1-(3-ethoxyphenyl)ethanamine as the amine. The title compound was obtained in 17% yield as the faster eluting isomer.

¹H NMR (300 MHz, CDCl₃) δ 7.26-7.11 (m, 5H), 6.91-6.84 (m, 2H), 6.77 (ddd, J=8.3, 2.5, 0.9 Hz, 1H), 4.13 (q, J=7.1 Hz, 2H), 4.04 (q, J=7.0 Hz, 2H), 3.81 (q, J=6.6 Hz, 1H), 3.55 (s, 2H), 3.13-3.00 (m, 1H), 2.97-2.83 (m, 1H), 2.23-2.12 (m, 1H), 2.06-1.89 (m, 2H), 1.81-1.50 (m, 2H), 1.47-1.31 (m, 7H), 1.24 (t, J=7.2 Hz, 3H).

### Intermediate 6: {4-[(1R,3S)-3-((1R)-1-Benzol[1,3]dioxol-4-yl-ethylamino)-cyclo-pentyl]-phenyl}-acetic acid ethyl ester

The procedure described for intermediate 4 was followed using Intermediate 1 as the ketone and (R)-1-(2H-benzo[d]1,3-dioxolen-4-yl)-ethylamine hydrochloride as the amine. The title compound was obtained in 23% yield as the faster eluting isomer.

¹H NMR (300 MHz, CDCl₃) δ 7.21-7.10 (m, 4H), 6.83-6.74 (m, 2H), 6.72 (dd, J=6.1, 2.9 Hz, 1H), 5.93 (s, 2H), 4.13 (q, J=7.2 Hz, 2H), 3.96 (q, J=6.7 Hz, 1H), 3.55 (s, 2H), 3.15-3.01 (m, 1H), 3.00-2.85 (m, 1H), 2.23-2.11 (m, 1H), 2.06-1.54 (m, 5H), 1.52-1.35 (m, 4H), 1.24 (t, J=7.1 Hz, 3H).

### Intermediate 7: (4-{(1R,3S)-3-[(1R)-1-(3-Chloro-phenyl)-ethylamino]-cyclo-pentyl}-phenyl)-acetic acid

Intermediate 4 (0.19 mmol) was dissolved in 2 mL of THF-MeOH (1:1) and treated with 4M NaOH (0.5 mL) for 2 h at rt. The solvent was evaporated, the pH was adjusted to approx. 5-6 with 1M HCl, and the mixture was extracted with DCM. The combined organic extracts were dried, filtered and concentrated in vacuo affording the title compound in quantitative yield as a colorless solid. LC/MS: M=357; RT=2.92.

### Intermediate 8: (4-{(1R,3S)-3-[(1R)-1-(3-Ethoxy-phenyl)-ethylamino]-cyclo-pentyl}-phenyl)-acetic acid

Intermediate 5 (0.19 mmol) was dissolved in 2 mL of THF-MeOH (1:1) and treated with 4M NaOH (0.5 mL) for 2 h at rt. The solvent was evaporated, the pH was adjusted to approx. 5-6 with 1M HCl, and the mixture was extracted with DCM. The combined organic extracts were dried, filtered and concentrated in vacuo affording the title compound in 95% yield as a colorless solid. LC/MS: M=367; RT=2.82.

### Intermediate 9: 2-[4-[(1R,3S)-3-[[(1R)-1-(1,3-benzodioxol-4-yl)ethyl]amino]-cyclopentyl]-phenyl]acetic acid

Intermediate 6 (0.19 mmol) was dissolved in 2 mL of THF-MeOH (1:1) and treated with 4M NaOH (0.5 mL) for 2 h at rt. The solvent was evaporated, the pH was adjusted to approx. 5-6 with 1M HCl, and the mixture was extracted with DCM. The combined organic extracts were dried, filtered and concentrated in vacuo affording the title compound in quantitative yield as a colorless solid.

¹H NMR (300 MHz, CDCl₃) δ 9.10 (br s, 1H), 7.35 (d, J=7.9 Hz, 1H), 7.05 (d, J=8.0 Hz, 2H), 6.95-6.85 (m, 3H), 6.80 (dd, J=7.7, 0.9 Hz, 1H), 5.97 (dd, J=12.2, 1.1 Hz, 2H), 4.50 (q, J=6.6 Hz, 1H), 3.53-3.39 (m, 2H), 3.24-3.08 (m, 1H), 2.76-2.60 (m, 1H), 2.15-1.81 (m, 4H), 1.81-1.66 (m, 4H), 1.52-1.34 (m, 1H).

### Example 1

### 4-[2-[4-[(1R,3S)-3-[[(1R)-1-(4-Fluoro-3-methoxy-phenyl)ethyl]-amino]-cyclopentyl]phenyl]acetyl]piperazin-2-one; formic acid (Compound 101)

General procedure B was followed using Intermediate 3 as the acid and piperazin-2-one as the amine.

¹H NMR (300 MHz, DMSO) δ 8.25 (5, 1H), 8.09/8.01 (5, 1H, rotamers), 7.26 (dd, J=8.5, 1.8 Hz, 1H), 7.20-7.07 (m, 5H), 6.96 (ddd, J=8.2, 4.4, 1.9 Hz, 1H), 4.08-3.89 (m, 3H), 3.84 (s, 3H), 3.73-3.55 (m, 4H), 3.18-2.97 (m, 3H), 2.96-2.80 (m, 1H), 2.20-2.05 (m, 1H), 1.97-1.60 (m, 4H), 1.53-1.38 (m, 1H), 1.35 (d, J=6.6 Hz, 3H).

### Example 2

### 2-[4-[(1R,3S)-3-[[(1R)-1-(4-Fluoro-3-methoxy-phenyl)ethyl]amino]-cyclopentyl]phenyl]-N-[2-(methanesulfonamido)ethyl]acetamide; formic acid (Compound 102)

General procedure B was followed using Intermediate 3 as the acid and N-(2-aminoethyl)methanesulfonamide hydrochloride as the amine.

¹H NMR (600 MHz, DMSO) δ 8.31 (s, 1H), 8.13 (t, J=5.8 Hz, 1H), 7.30 (dd, J=8.4, 1.8 Hz, 1H), 7.20-7.12 (m, 5H), 7.09 (t, J=5.4 Hz, 1H), 6.99 (ddd, J=8.2, 4.2, 2.0 Hz, 1H), 4.03 (q, J=6.6 Hz, 1H), 3.84 (s, 3H), 3.36 (s, 2H), 3.15 (dd, J=12.7, 6.4 Hz, 2H), 3.12-3.06 (m, 1H), 2.98 (dd, J=12.1, 6.3 Hz, 2H), 2.92-2.84 (m, 1H), 2.87 (s, 3H), 2.17-2.10 (m, 1H), 1.94-1.84 (m, 2H), 1.81-1.73 (m, 1H), 1.72-1.64 (m, 1H), 1.50 (td, J=11.9, 9.4 Hz, 1H), 1.39 (d, J=6.7 Hz, 3H).

### Example 3

### 2-[4-[(1R,3S)-3-[[(1R)-1-(4-Fluoro-3-methoxy-phenyl)ethyl]amino]-cyclopentyl]phenyl]-N-(2-sulfamoylethyl)acetamide; formic acid (Compound 103)

General procedure B was followed using Intermediate 3 as the acid and 2-amino-ethanesulfonic acid amide hydrochloride as the amine.

¹H NMR (300 MHz, DMSO) δ 8.26 (s, 1H), 8.15 (t, J=5.7 Hz, 1H), 7.25 (dd, J=8.5, 1.9 Hz, 1H), 7.20-7.09 (m, 5H), 6.95 (ddd, J=8.2, 4.4, 2.0 Hz, 1H), 6.88 (br s, 2H), 3.95 (q, J=6.5 Hz, 1H), 3.84 (s, 3H), 3.47-3.37 (m, 2H), 3.35 (s, 2H), 3.14-2.98 (m, 3H), 2.96-2.80 (m, 1H), 2.18-2.05 (m, 1H), 1.96-1.60 (m, 4H), 1.51-1.37 (m, 1H), 1.34 (d, J=6.6 Hz, 3H).

### Example 4

### 2-[4-[(1R,3S)-3-[[(1R)-1-(1,3-Benzodioxol-4-yl)ethyl]amino]-cyclo-pentyl]-phenyl]-N-(2-sulfamoylethyl)acetamide (Compound 104)

General procedure C was followed using Intermediate 9 as the acid and 2-amino-ethanesulfonic acid amide hydrochloride as the amine.

¹H NMR (600 MHz, DMSO) δ 8.15 (t, J=5.7 Hz, 1H), 7.17-7.10 (m, 4H), 6.92 (dd, J=7.9, 1.1 Hz, 1H), 6.88 (s, 2H), 6.80 (dd, J=9.8, 5.7 Hz, 1H), 6.76 (dd, J=7.7, 1.2 Hz, 1H), 5.97 (d, J=0.4 Hz, 2H), 3.92 (q, J=6.7 Hz, 1H), 3.45-3.38 (m, 2H), 3.35 (s, 2H), 3.12-3.07 (m, 2H), 2.98-2.91 (m, 1H), 2.90-2.82 (m, 1H), 2.10-2.02 (m, 1H), 1.91-1.84 (m, 1H), 1.82-1.74 (m, 1H), 1.68-1.53 (m, 2H), 1.34-1.27 (m, 1H), 1.26 (d, J=6.7 Hz, 3H).

### Example 5

### 2-[4-1(1R,3S)-3-[[(1R)-1-(3-Chlorophenyl)ethyl]amino]cyclopentyl]phenyl]-N-(2-sulfamoylethyl)acetamide (Compound 105)

General procedure C was followed using Intermediate 7 as the acid and 2-amino-ethanesulfonic acid amide hydrochloride as the amine.

¹H NMR (600 MHz, DMSO) δ 8.15 (t, J=5.7 Hz, 1H), 7.44-7.41 (m, 1H), 7.35-7.29 (m, 2H), 7.27-7.23 (m, 1H), 7.16-7.11 (m, 4H), 6.88 (s, 2H), 3.78 (q, J=6.6 Hz, 1H), 3.44-3.39 (m, 2H), 3.35 (s, 2H), 3.09 (dd, 2H), 2.91-2.80 (m, 2H), 2.26 (br s, 1H), 2.06-1.99 (m, 1H), 1.91-1.84 (m, 1H), 1.81-1.73 (m, 1H), 1.68-1.53 (m, 2H), 1.30 (td, j=11.8, 8.7 Hz, 1H), 1.23 (d, J=6.6 Hz, 3H).

### Example 6

### 2-[4-[(1R,3S)-3-[[(1R)-1-(3-Ethoxyphenyl)ethyl]amino]cyclopentyl]phenyl]-N-(2-sulfamoylethyl)acetamide (Compound 106)

General procedure C was followed using Intermediate 8 as the acid and 2-amino-ethanesulfonic acid amide hydrochloride as the amine.

¹H NMR (600 MHz, DMSO) δ 8.15 (br t, 1H), 7.18 (t, J=7.8 Hz, 1H), 7.16-7.11 (m, 4H), 6.92-6.90 (m, 1H), 6.88 (d, J=7.6 Hz, 1H), 6.85 (s, 2H), 6.75-6.72 (m, 1H), 4.00 (q, 2H), 3.72 (p, J=6.4 Hz, 1H), 3.44-3.38 (m, 2H), 3.34 (s, 2H), 3.09 (dd, J=8.2, 6.4 Hz, 2H), 2.93-2.79 (m, 2H), 2.10 (t, J=6.3 Hz, 1H), 2.05-1.99 (m, 1H), 1.90-1.83 (m, 1H), 1.80-1.73 (m, 1H), 1.67-1.53 (m, 2H), 1.31 (t, J=7.0 Hz, 3H), 1.32-1.26 (m, 1H), 1.22 (d, J=6.6 Hz, 3H).

### Example 7

### 4-[2-[4-[(1R,3S)-3-[[(1R)-1-(3-Ethoxyphenyl)ethyl]amino]cyclopentyl]-phenyl]acetyl]piperazin-2-one (Compound 107)

General procedure C was followed using Intermediate 8 as the acid and piperazin-2-one as the amine.

¹H NMR (600 MHz, DMSO) δ 8.11/8.02 (s, 1H, rotamers), 7.21-7.07 (m, 5H), 6.92-6.90 (m, 1H), 6.88 (d, J=7.6 Hz, 1H), 6.75-6.72 (m, 1H), 4.05/3.93 (s, 2H, rotamers), 4.00 (q, J=7.0 Hz, 2H), 3.72 (q, J=6.6 Hz, 1H), 3.70-3.58 (m, 4H), 3.17-3.10 (m, 2H), 2.92-2.80 (m, 2H), 2.12 (br s, 1H), 2.06-2.00 (m, 1H), 1.91-1.84 (m, 1H), 1.80-1.73 (m, 1H), 1.68-1.53 (m, 2H), 1.34-1.27 (m, 4H), 1.22 (d, J=6.6 Hz, 3H).

### Example 8

### 2-[4-[(1R,3S)-3-[[(1R)-1-(4-Fluoro-3-methoxy-phenyl)ethyl]amino]-cyclopentyl]phenyl]-N-[2-(2-hydroxyethylamino)ethyl]acetamide (Compound 108)

General procedure B was followed using Intermediate 3 as the acid and N-(2-hydroxyethyl)ethylendiamine as the amine.

¹H NMR (600 MHz, DMSO) δ 7.95 (t, J=5.5 Hz, 1H), 7.17-7.14 (m, 1H), 7.13 (m, 4H), 7.09 (dd, J=11.5, 8.2 Hz, 1H), 6.88 (ddd, J=8.2, 4.4, 1.9 Hz, 1H), 4.44 (t, J=5.0 Hz, 1H), 3.82 (s, 3H), 3.75 (q, J=6.5 Hz, 1H), 3.42 (dd, J=10.1, 4.8 Hz, 2H), 3.36-3.31 (m, 2H, overlapping residual water peak), 3.09 (q, J=6.4 Hz, 2H), 2.92-2.80 (m, 2H), 2.57-2.53 (m, 4H), 2.06-2.00 (m, 1H), 1.91-1.84 (m, 1H), 1.82-1.75 (m, 1H), 1.68-1.53 (m, 2H), 1.30 (m, 1H), 1.23 (d, J=6.6 Hz, 3H).

### Example 9

### 2-[4-[(1R,3S)-3-[[(1R)-1-(4-Fluoro-3-methoxy-phenyl)ethyl]amino]-cyclopentyl]phenyl]-1-(3-hydroxyazetidin-1-yl)ethanone (Compound 109)

General procedure B was followed using Intermediate 3 as the acid and 3-hydroxyazetidine hydrochloride as the amine.

¹H NMR (600 MHz, DMSO) δ 7.18-7.07 (m, 6H), 6.88 (ddd, J=8.1, 4.4, 1.9 Hz, 1H), 5.70 (d, J=6.0 Hz, 1H), 4.45-4.39 (m, 1H), 4.35-4.30 (m, 1H), 4.03-3.98 (m, 1H), 3.86 (dd, J=9.0, 4.3 Hz, 1H), 3.82 (s, 3H), 3.75 (q, J=6.5 Hz, 1H), 3.55 (dd, J=10.1, 4.5 Hz, 1H), 3.34 (s, 2H, overlapping residual water peak), 2.92-2.80 (m, 2H), 2.07-2.01 (m, 1H), 1.91-1.84 (m, 1H), 1.82-1.75 (m, 1H), 1.68-1.53 (m, 2H), 1.31 (td, J=11.8, 8.8 Hz, 1H), 1.23 (d, J=6.6 Hz, 3H).

### Example 10

### 1-(1,4-Diazepan-1-yl)-2-[4-[(1R,3S)-3-[[(1R)-1-(4-fluoro-3-methoxy-Phenyl)ethyl]amino]cyclopentyl]phenyl]ethanone (Compound 110)

General procedure B was followed using Intermediate 3 as the acid and 1,4-diazepane as the amine.

¹H NMR (600 MHz, DMSO) δ 7.19-7.07 (m, 6H), 6.88 (ddd, J=8.2, 4.4, 1.9 Hz, 1H), 3.82 (s, 3H), 3.75 (q, J=6.5 Hz, 1H), 3.62/3.60 (s, 2H, rotamers), 3.50 (t, J=6.2 Hz, 1H), 3.48-3.45 (m, 1H), 3.45-3.41 (m, 1H), 3.40 (dd, J=6.1, 4.5 Hz, 1H), 2.91-2.81 (m, 2H), 2.71-2.67 (m, 2H), 2.65-2.60 (m, 2H), 2.07-2.01 (m, 1H), 1.91-1.84 (m, 1H), 1.82-1.75 (m, 1H), 1.68-1.53 (m, 4H), 1.31 (td, J=11.7, 8.8 Hz, 1H), 1.23 (d, J=6.6 Hz, 3H).

### Example 11

### N-(2-Aminoethyl)-2-[4-[(1R,3S)-3-[[(1R)-1-(4-fluoro-3-methoxy-phenyl)ethyl]amino]cyclopentyl]phenyl]acetamide (Compound 111)

General procedure A was followed using Intermediate 3 as the acid and N-(tert-butoxycarbonyl)-1,2-diaminoethane as the amine.

¹H NMR (600 MHz, DMSO) δ 8.06 (t, J=5.5 Hz, 1H), 7.19-7.06 (m, 6H), 6.88 (ddd, J=8.2, 4.3, 1.9 Hz, 1H), 3.82 (s, 3H), 3.76 (q, J=6.5 Hz, 1H), 3.23-3.14 (m, 2H), 3.12 (dd, J=12.2, 6.3 Hz, 2H), 2.93-2.80 (m, 2H), 2.68 (dd, J=12.9, 6.4 Hz, 2H), 2.06-2.00 (m, 1H), 1.91-1.84 (m, 1H), 1.84-1.75 (m, 1H), 1.68-1.54 (m, 2H), 1.31 (td, J=11.7, 8.9 Hz, 1H), 1.24 (d, J=6.6 Hz, 3H).

### Example 12

### 1-(4-Amino-1-piperidyl)-2-[4-[(1R,3S)-3-[[(1R)-1-(4-fluoro-3-methoxy-phenyl)ethyl]amino]cyclopentyl]phenyl]ethanone (Compound 112)

General procedure A was followed using Intermediate 3 as the acid and 4-(tert-butoxycarbonylamino)piperidine as the amine.

¹H NMR (600 MHz, DMSO) δ 7.17-7.13 (m, 3H), 7.12-7.07 (m, 3H), 6.88 (ddd, J=8.2, 4.4, 1.9 Hz, 1H), 4.19-4.14 (m, 1H), 3.85-3.79 (m, 4H), 3.75 (d, J=6.6 Hz, 1H), 3.66-3.58 (m, 2H), 3.35-3.28 (m, 1H, overlapping residual water peak), 3.02-2.96 (m, 1H), 2.92-2.80 (m, 2H), 2.76-2.65 (m, 1H), 2.07-2.01 (m, 1H), 1.93-1.53 (m, 6H), 1.30 (dd, J=11.7, 8.8 Hz, 1H), 1.23 (d, J=6.6 Hz, 3H), 1.05-0.94 (m, 2H).

### Example 13

### 2-[4-[(1R,3S)-3-[[(1R)-1-(4-Fluoro-3-methoxy-phenyl)ethyl]amino]-cyclopentyl]phenyl]-1-piperazin-1-yl-ethanone (Compound 113)

General procedure A was followed using Intermediate 3 as the acid and 1-(tert-butoxycarbonyl)piperazine as the amine.

¹H NMR (600 MHz, DMSO) δ 7.17-7.13 (m, 3H), 7.12-7.07 (m, 3H), 6.88 (ddd, J=8.2, 4.4, 1.9 Hz, 1H), 3.82 (s, 3H), 3.75 (q, J=6.6 Hz, 1H), 3.62 (s, 2H), 3.37-3.28 (m, 4H, overlapping residual water peak), 2.92-2.80 (m, 2H), 2.60-2.55 (m, 2H), 2.55-2.51 (m, 2H), 2.08-2.01 (m, 1H), 1.92-1.84 (m, 1H), 1.82-1.74 (m, 1H), 1.68-1.53 (m, 2H), 1.30 (td, J=11.7, 8.8 Hz, 1H), 1.23 (d, J=6.6 Hz, 3H).

### Example 14

### 2-[4-[(1R,3S)-3-[[(1R)-1-(4-Fluoro-3-methoxy-phenyl)ethyl]amino]-cyclopentyl]phenyl]-N-(3-sulfamoylpropyl)acetamide (Compound 114)

General procedure A was followed using Intermediate 3 as the acid and 3-aminopropane-1-sulfonamide hydrochloride as the amine.

¹H NMR (600 MHz, DMSO) δ 8.10 (t, J=5.7 Hz, 1H), 7.19-7.06 (m, 6H), 6.91-6.85 (m, 1H), 6.79 (s, 2H), 3.82 (s, 3H), 3.75 (q, J=6.5 Hz, 1H), 3.34 (m, 2H, overlapping residual water peak), 3.13 (q, J=6.7 Hz, 2H), 2.98-2.92 (m, 2H), 2.92-2.79 (m, 2H), 2.06-2.00 (m, 1H), 1.92-1.84 (m, 1H), 1.84-1.74 (m, 3H), 1.68-1.52 (m, 2H), 1.31 (td, J=11.8, 8.8 Hz, 1H), 1.23 (d, J=6.6 Hz, 3H).

1. A compound of general formula I 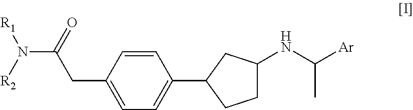
wherein
Ar represents phenyl or C₁₋₅heterocycloalkylphenyl, wherein said phenyl is optionally substituted with one or more, same or different substituents independently selected from halogen, hydroxy, C₁₋₆alkyl, trifluoromethyl or C₁₋₄alkoxy;
R₁ represents hydrogen, or is selected from the group consisting of C₁₋₆alkyl, C₂₋₆alkenyl, C₂₋₆alkynyl, hydroxyC₂₋₆alkyl, aminoC₂₋₆alkyl, hydroxyC₂₋₆alkylaminoC₂₋₆alkyl, C₁₋₃ alkylsulfonyl aminoC₁₋₆alkyl, aminosulfonyl C₁₋₆alkyl, aminocarbonylC₁₋₆alkyl, or C_{1- 5}heterocycloalkyl comprising 1-4 hetero atoms selected from N, O and S, wherein said C₁₋₆alkyl, C₂₋₆alkenyl, C₂₋₆alkynyl, hydroxyC₂₋₆alkyl, aminoC₂₋₆alkyl, hydroxyC₂₋₆alkylaminoC₁₋₆alkyl, C₁₋₃alkylsulfonylaminoC₂₋₆alkyl, aminosulfonylC₁₋₆alkyl, aminocarbonylC₁₋₆alkyl, or C₁₋₅heterocycloalkyl comprising 1-4 hetero atoms selected from N, O and S, is optionally further substituted by one or more substituents selected from halogen, hydroxy, trifluoromethyl, C₁₋₃alkylsulfonylamino or —NH₂;
R₂ represents hydrogen, or is selected from the group consisting of C₁₋₆alkyl, C₂₋₆alkenyl, aminoC₂₋₆alkyl, C₃₋₇cycloalkyl, or C₁₋₅heterocycloalkyl comprising 1-4 hetero atoms selected from N, O and S;
provided one of R₁ and R₂ is not hydrogen;
or R₁ and R₂ together with the adjacent nitrogen to which they are attached form a 4, 5, 6 or 7-membered C₁₋₆heterocycloalkyl comprising one or more heteroatoms selected from the group consisting of O, S and N, said C₁₋₆heterocycloalkyl being optionally substituted by oxo, hydroxy, halogen, trifluoromethyl, C₁₋₆alkyl, —NH₂, —S(O)₂NH₂, —S(O)₂CH₃, C₁₋₆alkylcarbonyl, hydroxyC₂₋₆alkyl, C₁₋₆alkoxy, aminoC₂₋₆alkyl, C₁₋₆alkylamino, or aminosulfonylC₁₋₆alkylamino;
as well as stereoisomers, and pharmaceutically acceptable salts thereof.

2. A compound according to claim 1, represented by formula Ia or Ib

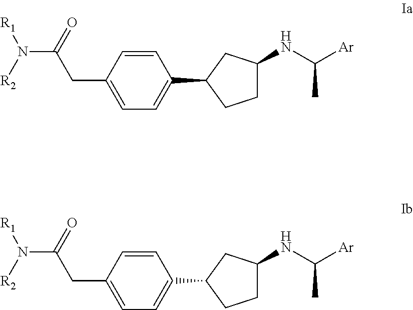

3. A compound according to claim 1, wherein Ar represents phenyl optionally substituted with one or more, same or different substituents, independently selected from chloro, fluoro or C₁₋₃alkoxy.

4. A compound according to claim 3, wherein Ar represents 4-fluoro-3-methoxyphenyl, 3-chlorophenyl, or 3-ethoxyphenyl.

5. A compound according to claim 1, wherein Ar represents C₂₋₅heterocycloalkylphenyl comprising 1-3 hetero atoms selected from O.

6. A compound according to claim 1, wherein R₁ is selected from
the group consisting of C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₋₄alkyl, aminosulfonylC₁₋₄alkyl, aminocarbonylC₁₋₄alkyl, or C₃₋₅heterocycloalkyl comprising 1-2 hetero atoms selected from N, O and S,
wherein said C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₋₄alkyl, aminosulfonylC₁₋₄alkyl, aminocarbonylC₁₋₄alkyl, or C₃₋₅heterocycloalkyl comprising 1-2 hetero atoms selected from N, O and S, is optionally further substituted by one or more substituents selected from halogen, hydroxy, C₁₋₂alkylsulfonylamino or —NH₂.

7. A compound according to claim 1, wherein R₁ is selected from methylsulfonylaminoethyl, aminosulfonylethyl, aminosulfonylpropyl, hydroxyethylaminoethyl or aminoethyl.

8. A compound according to claim 1, wherein R₂ represents hydrogen.

9. A compound according claim 1, wherein R₁ or R₂ together with the nitrogen to which they are attached form a 4, 5, 6 or 7 membered C₃₋₆heterocycloalkyl comprising one or two heteroatoms selected from the group consisting of O, S and N, said C₃₋₆heterocycloalkyl being optionally substituted by oxo, hydroxy, trifluoromethyl, C₁₋₆alkyl, —NH₂, —S(O)₂NH₂, —S(O)₂CH₃, C₁₋₆alkylcarbonyl, hydroxyC₂₋₆alkyl, C₁₋₆alkoxy or C₂₋₆alkylamino.

10. A compound according to claim 9, wherein the heterocyclic ring is selected from the group consisting of piperazinyl, piperidinyl, azetidinyl, or diazepanyl, optionally substituted with oxo, hydroxy, or NH₂.

11. A compound according to claim 1 selected from the group comprising:
4-[2-[4-[(1R,3S)-3-[[(1R)-1-(4-fluoro-3-methoxy-phenyl)ethyl]amino]cyclopentyl]-phenyl]acetyl]piperazin-2-one; formic acid (compound 101),
2-[4-[(1R,3S)-3-[[(1R)-1-(4-fluoro-3-methoxy-phenyl)ethyl]amino]cyclopentyl]phenyl]-N-[2-(methanesulfonamido)ethyl]acetamide; formic acid (compound 102),
2-[4-[(1R,3S)-3-[[(1R)-1-(4-fluoro-3-methoxy-phenyl)ethyl]amino]cyclopentyl]phenyl]-N-(2-sulfamoylethyl)acetamide; formic acid (compound 103),
2-[4-[(1R,3S)-3-[[(1R)-1-(1,3-benzodioxol-4-yl)ethyl]amino]cyclopentyl]phenyl]-N-(2-sulfamoylethyl)acetamide (compound 104),
2-[4-[(1R,3S)-3-[[(1R)-1-(3-chlorophenyl)ethyl]amino]cyclopentyl]phenyl]-N-(2-sulfamoylethyl)acetamide (compound 105),
2-[4-[(1R,3S)-3-[[(1R)-1-(3-ethoxyphenyl)ethyl]amino]cyclopentyl]phenyl]-N-(2-sulfamoylethyl)acetamide (compound 106),
4-[2-[4-[(1R,3S)-3-[[(1R)-1-(3-ethoxyphenyl)ethyl]amino]cyclopentyl]phenyl]-acetyl]piperazin-2-one (compound 107),
2-[4-[(1R,3S)-3-[[(1R)-1-(4-fluoro-3-methoxy-phenyl)ethyl]amino]cyclopentyl]phenyl]-N-[2-(2-hydroxyethylamino)ethyl]acetamide (compound 108),
2-[4-[(1R,3S)-3-[[(1R)-1-(4-fluoro-3-methoxy-phenyl)ethyl]amino]cyclopentyl]phenyl]-1-(3-hydroxyazetidin-1-yl)ethanone (compound 109),
1-(1,4-diazepan-1-yl)-2-[4-[(1R,3S)-3-[[(1R)-1-(4-fluoro-3-methoxy-phenyl)ethyl]amino]-cyclopentyl]phenyl]ethanone (compound 110),
N-(2-aminoethyl)-2-[4-[(1R,3S)-3-[[(1R)-1-(4-fluoro-3-methoxy-phenyl)ethyl]amino]-cyclopentyl]phenyl]acetamide (compound 111),
1-(4-amino-1-piperidyl)-2-[4-[(1R,3S)-3-[[(1R)-1-(4-fluoro-3-methoxy-phenyl)ethyl]-amino]cyclopentyl]phenyl]ethanone (compound 112),
2-[4-[(1R,3S)-3-[[(1R)-1-(4-fluoro-3-methoxy-phenyl)ethyl]amino]cyclopentyl]phenyl]-1-piperazin-1-yl-ethanone (compound 113), or
2-[4-[(1R,3S)-3-[[(1R)-1-(4-fluoro-3-methoxy-phenyl)ethyl]amino]cyclopentyl]phenyl]-N-(3-sulfamoylpropyl)acetamide (compound 114).

12. A compound according to claim 1 for use as a medicament in therapy.

13. A compound according to claim 1 for use in the treatment, amelioration or prophylaxis of physiological disorders or diseases associated with disturbances of CaSR activity.

14. A pharmaceutical composition comprising a compound according to claim 1 or a pharmaceutically acceptable salt, solvate, hydrate or in vivo hydrolysable ester thereof together with a pharmaceutically acceptable vehicle or excipient.

15. A method of preventing, treating or ameliorating parathyroid carcinoma, parathyroid adenoma, primary parathyroid hyperplasia, cardiac, renal or intestinal dysfunctions, diseases of the central nervous system, chronic renal failure, chronic kidney disease, polycystic kidney disorder, podocyte-related diseases, primary hyperparathyroidism, secondary hyperparathyroidism, tertiary hyperparathyroidism, anemia, cardiovascular diseases, renal osteodystrophy, osteitis fibrosa, adynamic bone disease, osteoporosis, steroid induced osteoporosis, senile osteoporosis, post-menopausal osteoporosis, osteomalacia and related bone disorders, bone loss post renal transplantation, cardiovascular diseases, gastrointestinal diseases, endocrine and neurodegenerative diseases, cancer, Alzheimer's disease, IBS, IBD, malassimilation, malnutrition, abnormal intestinal motility such as diarrhea, vascular calcification, abnormal calcium homeostasis, hypercalcemia, or renal bone diseases, the method comprising administering to a patient in need thereof an effective amount of a compound according to any one of claims 1-11, optionally in combination or as supplement with an active vitamin-D sterol or vitamin-D derivative, such as 1-α-hydroxycholecalciferol, ergocalciferol, cholecalciferol, 25-hydroxycholecalciferol, 1-α-25-dihydroxycholecalciferol, or in combination or as supplement with phosphate binders, estrogens, calcitonin or biphosphonates.

16. A compound selected from the group consisting of
Ethyl 2-[4-[(1R)-3-oxocyclopentyl]phenyl]acetate (Intermediate 1),
Ethyl 2-[4-[(1R,3S)-3-[[(1R)-1-(4-fluoro-3-methoxy-phenyl)ethyl]amino]-cyclopentyl]phenyl]acetate (Intermediate 2),
2-[4-[(1R,3S)-3-[[(1R)-1-(4-fluoro-3-methoxy-phenyl)ethyl]amino]cyclopentyl]phenyl]acetic acid (Intermediate 3),
(4-{(1R,3S)-3-[(1R)-1-(3-Chloro-phenyl)-ethylamino]-cyclopentyl}-phenyl)-acetic acid ethyl ester (Intermediate 4),
(4-{(1R,3S)-3-[(1R)-1-(3-Ethoxy-phenyl)- ethylamino]-cyclopentyl}-phenyl)-acetic acid ethyl ester (Intermediate 5),
{4-[(1R,3S)-3-((1R)-1-Benzo[1,3]dioxol-4-yl-ethylamino)-cyclopentyl]-phenyl}-acetic acid ethyl ester (Intermediate 6),
(4-{(1R,3S)-3-[(1R)-1-(3-Chloro-phenyl)-ethylamino]-cyclopentyl}-phenyl)-acetic acid (Intermediate 7),
(4-{(1R,3S)-3-[(1R)-1-(3-Ethoxy-phenyl)-ethylamino]-cyclopentyl}-phenyl)-acetic acid (Intermediate 8), or
2-[4-[(1R,3S)-3-[[(1R)-1-(1,3-benzodioxol-4-yl)ethyl]amino]cyclopentyl]phenyl]acetic acid (Intermediate 9).

17. A compound according to claim 2, wherein Ar represents phenyl optionally substituted with one or more, same or different substituents, independently selected from chloro, fluoro or C₁₋₃alkoxy.

18. A compound according to claim 2, wherein Ar represents C₂₋₅heterocycloalkylphenyl comprising 1-3 hetero atoms selected from O.

19. A compound according to claim 2, wherein R₁ is selected from the group consisting of C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₋₄alkyl, aminosulfonylC₁₋₄alkyl, aminocarbonylC₁₋₄alkyl, or C₃₋₅heterocycloalkyl comprising 1-2 hetero atoms selected from N, O and S,
wherein said C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₄alkyl, aminosulfonylC₁₋₄alkyl, aminocarbonylC₁₋₄alkyl, or C₃₋₅heterocycloalkyl comprising 1-2 hetero atoms selected from N, O and S, is optionally further substituted by one or more substituents selected from halogen, hydroxy, C₁₋₂alkylsulfonylamino or —NH₂,

20. A compound according to claim 3, wherein R₁ is selected from the group consisting of C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₋₄alkyl, aminosulfonylC₁₋₄alkyl, aminocarbonylC₁₋₄alkyl, or C₃₋₅heterocycloalkyl comprising 1-2 hetero atoms selected from N, O and S,
wherein said C₁₋₄alkyl, C₂₋₄alkenyl, C₂₋₄alkynyl, hydroxyC₂₋₄alkyl, aminoC₂₋₄alkyl, hydroxyC₂₋₄alkylaminoC₂₋₄alkyl, C₁₋₂alkylsulfonylaminoC₂₋₄alkyl, aminosulfonylC₁₋₄alkyl, aminocarbonylC₁₋₄alkyl, or C₃₋₅heterocycloalkyl comprising 1-2 hetero atoms selected from N, O and S, is optionally further substituted by one or more substituents selected from halogen, hydroxy, C₁₋₂alkylsulfonylamino or —NH₂.
